(19) United States
(12) Patent Application Publication
(10) Pub. No.: US 2010/0297030 A1
(43) Pub. Date: Nov. 25, 2010

(54) Composition, device, and method for treating sexual dysfunction via inhalation

(75) Inventors: John Nicholas Staniforth, Bath (GB); David Alexander Vodden Morton, Bath (GB); Michael Tobyn, Trowbridge (GB); Stephen Eason, Redgrave (GB); Quentin Harmer, Cambridge (GB); David Ganderton, Bishop (GB)

Correspondence Address: Davidson, Davidson & Kappel, LLC, 485 7th Avenue, 14th Floor, New York, NY, 10018, US

(73) Assignee: Vectura Drug Delivery, Chippenham (GB)

(21) Appl. No.: 12/653,179

(22) Filed: Dec. 9, 2009

Related U.S. Application Data
(63) Continuation of application No. 10/621,964, filed on Jul. 17, 2003, now abandoned.
Application No. 10/621,964 is a continuation-in-part of application No. 10/413,022, filed on Apr. 14, 2003, now abandoned.

(51) Int. Cl. A61K 9/12; A61K 31/473; A61P 15/10; A61P 15/00

(52) U.S. Cl. 424/43; 514/289

(57) ABSTRACT

A composition, device, and method for treating sexual dysfunction via inhalation is provided which comprises inhaling a dose of apomorphine or pharmaceutically acceptable salt(s) or ester(s) thereof.

This application is a continuation-in-part of U.S. patent application Ser. No. 10/413,022, filed Apr. 14, 2003, entitled “Composition, Device, And Method For Treating Sexual Dysfunction Via Inhalation”, the entire disclosure of which is hereby incorporated by reference.

### BACKGROUND OF THE INVENTION

The term “erectile dysfunction” has been defined by the National Institutes of Health as the inability of the male to attain and maintain erection of the penis sufficient to permit satisfactory sexual intercourse. See J. Am. Med. Assoc., 270(1):83-90 (1993). Because adequate arterial blood supply is critical for erection, any disorder that impairs blood flow may be implicated in the etiology of erectile failure. Erectile dysfunction affects millions of men and, although generally regarded as a benign disorder, has a profound impact on their quality of life. It is recognized, however, that in many men psychological desire, orgasmic capacity, and ejaculatory capacity are intact even in the presence of erectile dysfunction.

Etiological factors for erectile disorders have been categorized as psychogenic or organic in origin. Organic factors include those of a neurogenic origin and those of a vasculogenic origin. Neurogenic factors include, for example, lesions of the somatic nervous pathways which may impair reflexogenic erections and interrupt tactile sensations needed to maintain erections, and spinal cord lesions which, depending upon their location and severity, may produce varying degrees of erectile failure.

Psychogenic factors for erectile dysfunction include such processes as depression, anxiety, and relationship problems which can impair erectile functioning by reducing erotic focus or otherwise reducing awareness of sensory experience. This may lead to an inability to initiate or maintain an erection.

Vasculogenic risk factors include factors which affect blood flow and include cigarette smoking, diabetes mellitus, hypertension, alcohol, vascular disease, high levels of serum cholesterol, low levels of high-density lipoprotein (HDL), and other chronic disease conditions such as arthritis. The Massachusetts Male Aging Study (M:MAS, as reported by H. A. Feldman, et al., J. Urol., 151: 54-61 (1994) found, for example, that the age-adjusted probability of complete erectile dysfunction was three times greater in subjects reporting treated diabetes than in those without diabetes. While there is some disagreement as to which of the many aspects of diabetes is the direct cause of erectile dysfunction, vascular disease is most frequently cited.

The MMAS also found a significant correlation between erectile dysfunction and heart disease with two of its associated risk factors, hypertension and low serum high density lipoprotein (HDL). It has been reported that 8-10% of all untreated hypertensive patients are impotent at the time they are diagnosed with hypertension. The association of erectile dysfunction with vascular disease in the literature is strong, with impairments in the hemodynamics of erection demonstrated in patients with myocardial infarction, coronary bypass surgery, cerebrovascular accidents, and peripheral vascular disease. It also found cigarette smoking to be an independent risk factor for vasculogenic erectile dysfunction, with cigarette smoking found to exacerbate the risk of erectile dysfunction associated with cardiovascular diseases.

As described in U.S. Pat. Nos. 5,770,606 and 6,291,471, it is known to treat both psychogenic and organic erectile dysfunction in males with the opioid apomorphine. Two and three milligram sublingual tablets of apomorphine hydrochloride are currently available in Europe for the treatment of male erectile dysfunction under the name UPRIMA™ (see, e.g., European Public Assessment Report (EPAR) 1945).

Apomorphine is a derivative of morphine, and was first evaluated for use as a pharmacologic agent as an emetic in 1869. In the first half of the 20th century, apomorphine was used as a sedative for psychiatric disturbances and as a behavior-altering agent for alcoholics and addicts. By 1967, the dopaminergic effects of apomorphine were realized, and the compound underwent intensive evaluation for the treatment of Parkinsonism. Since that time, apomorphine has been classified as a selective dopamine receptor agonist that stimulates the central nervous system producing an arousal response manifested by yawning and penile erection in animals and man.

WO 01/74358 purports to describe a method for treatment of male erectile dysfunction using an inhaled apomophine formulation. The formulations exemplified therein comprise a solution of apomorphine and sodium metabisulfite in water, which are said to have been introduced directly into the lungs of a dog via the trachea.

U.S. Pat. No. 6,193,992 purports to describe a method of ameliorating sexual dysfunction in a human female which comprises administering to said human female apomorphine in an amount sufficient to increase intraclitoral blood flow and vaginal wall blood flow on stimulation of said female but less than the amount that induces substantial nausea

### SUMMARY OF THE INVENTION

In one aspect, the present invention is directed to methods for treating sexual dysfunction via inhalation therapy.

In accordance with one embodiment of the present invention, a method for treating sexual dysfunction via inhalation is provided which comprises inhaling a dose of from about 100 to about 1600 micrograms of apomorphine or pharmaceutically acceptable salt(s) or ester(s) thereof (based on the weight of the hydrochloride salt). Preferably, the dose comprises from about 200 micrograms to about 1600 micrograms of said apomorphine, more preferably, about 300 micrograms to about 1200 micrograms of said apomorphine, more preferably about 400 to about 1200 micrograms of said apomorphine. Most preferably, doses are provided in increments between 400 and 1200 micrograms, based upon the requirements and tolerance of the individual patients. For examples, doses may be provided of about 400, about 500, about 600, about 700, about 800, about 900, about 1000, about 1100, and/or about 1200 micrograms of said apomorphine.

In accordance with another embodiment of the present invention, a method for treating sexual dysfunction is provided which comprises inhaling a dose including apomorphine or a pharmaceutically acceptable salt or ester thereof, said dose being sufficient to provide a therapeutic effect in about 10 minutes or less.

Preferably, the dose of the above-referenced embodiments is a powder composition inhaled via a dry powder inhaler (“DPI”). However in other embodiments, the dose may be a solution or suspension formulation inhaled via a pressurized metered dose inhaler (“pMDI”).

In accordance with one such embodiment of the present invention, a method for treating sexual dysfunction via inhalation is provided which comprises inhaling a dose of a powder composition, the powder composition comprising apomorphine or pharmaceutically acceptable salt(s) or ester(s) thereof. Preferably, the powder composition further includes a carrier material, the carrier material has an average particle size of from about 40 to about 70 microns, and at least 90 percent of said apomorphine has a particle size of 5 microns or less.

In accordance with another embodiment of the present invention, a method for treating sexual dysfunction via inhalation is provided which comprises inhaling a dose of a powder composition, the dose of the powder composition comprising from about 100 micrograms to about 3200 micrograms of apomorphine or pharmaceutically acceptable salt(s) or ester(s) thereof (based on the weight of the hydrochloride salt). Preferably, the dose comprises from about 100 micrograms to about 1600 micrograms of said apomorphine, more preferably, about 200 micrograms to about 1600 micrograms of said apomorphine, more preferably, about 300 micrograms to about 1200 micrograms of said apomorphine, more preferably about 400 to about 1200 micrograms of said apomorphine. Most preferably, doses are provided in increments between 400 and 1200 micrograms, based upon the requirements and tolerance of the individual patients. For examples, doses may be provided of about 400, about 500, about 600, about 700, about 800, about 900, about 1000, about 100, and/or about 1200 micrograms of said apomorphine.

In accordance with another embodiment of the present invention, a method for treating sexual dysfunction via inhalation is provided which comprises inhaling a dose of a powder composition into the lungs of a patient, the dose of the powder composition delivering, in vitro, a fine particle dose of from about 100 micrograms to about 1600 micrograms of apomorphine or pharmaceutically acceptable salt(s) or ester(s) thereof (based on the weight of the hydrochloride salt), when measured by a Multistage Liquid Impinger, United States Pharmacopeia 26, Chapter 601 Apparatus 4 (2003). Preferably, the dose delivers, in vitro, a fine particle dose from about 200 micrograms to about 1000 micrograms of said apomorphine, more preferably, about 200 micrograms to about 800 micrograms of said apomorphine, more preferably, about 200 micrograms to about 600 micrograms of said apomorphine, and most preferably about 200 to about 400 micrograms of said apomorphine when measured by a Multistage Liquid Impinger, United States Pharmacopeia 26, Chapter 601 Apparatus 4 (2003).

In accordance with another embodiment of the present invention, a method for treating sexual dysfunction via inhalation is provided which comprises inhaling a dose of a powder composition, the powder composition comprising apomorphine or pharmaceutically acceptable salt(s) or ester(s) thereof and a carrier material, the carrier material having an average particle size of from about 40 to about 70 microns, at least 90 percent of said apomorphine having a particle size of 5 microns or less.

In another aspect, the present invention is directed to unit doses of apomorphine.

In accordance with one such embodiment of the present invention, a dose is provided which comprises a powder composition including a carrier material and from about 100 micrograms to about 3200 micrograms of apomorphine or a pharmaceutically acceptable salt or ester thereof (based on the weight of the hydrochloride salt). Preferably, the dose comprises from about 100 micrograms to about 1600 micrograms of said apomorphine, more preferably, about 200 micrograms to about 1600 micrograms of said apomorphine, more preferably, about 300 micrograms to about 1200 micrograms of said apomorphine, more preferably about 400 to about 1200 micrograms of said apomorphine. Most preferably, doses are provided in increments between 400 and 1200 micrograms, based upon the requirements and tolerance of the individual patients. For examples, doses may be provided of about 400, about 500, about 600, about 700, about 800, about 900, about 1000, about 100, and/or about 1200 micrograms of said apomorphine.

In accordance with another embodiment of the present invention, a dose is provided which comprises a powder composition including a carrier material and apomorphine or a pharmaceutically acceptable salt or ester thereof, the carrier material having an average particle size of from about 40 to about 70 microns, at least 90 percent of said apomorphine having a particle size of 5 microns or less.

In accordance with another embodiment of the present invention, a drug loaded blister is provided which comprises a base having a cavity formed therein, the cavity containing a powder composition including a carrier material and from about 100 micrograms to about 3200 micrograms of apomorphine or a pharmaceutically acceptable salt or ester thereof (based on the weight of the hydrochloride salt), the cavity having an opening which is sealed by a rupturable covering. Preferably, the powder composition comprises from about 100 micrograms to about 1600 micrograms of said apomorphine, more preferably, about 200 micrograms to about 1600 micrograms of said apomorphine, more preferably, about 300 micrograms to about 1200 micrograms of said apomorphine, more preferably about 400 to about 1200 micrograms of said apomorphine. Most preferably, doses are provided in increments between 400 and 1200 micrograms, based upon the requirements and tolerance of the individual patients. For examples, doses may be provided of about 400, about 500, about 600, about 700, about 800, about 900, about 1000, about 100, and/or about 1200 micrograms of said apomorphine.

In accordance with another embodiment of the present invention, a drug loaded blister is provided which comprises a base having a cavity formed therein, the cavity containing a powder composition including a carrier material and apomorphine or a pharmaceutically acceptable salt or ester thereof, the carrier material having an average particle size of from about 40 to about 70 microns, at least 90 percent of said apomorphine having a particle size of 5 microns or less, the enclosure having an open end, the cavity having an opening which is sealed by a rupturable covering.

In the above referenced embodiments, the doses and/or drug loaded blisters preferably include from 1 to 5 milligrams of powder composition, wherein apomorphine or its pharmaceutically acceptable salts comprise from about 3% to about 80%, preferably from about 5% to about 50%, and most preferably from about 15% to about 30% of the powder composition.

In another aspect, the present invention is directed to methods for producing an inhalable aerosol of a powdered apomorphine composition.

In accordance with one such embodiment, the method comprises entraining a powdered composition in a gas flow upstream from an inlet port of a vortex chamber having a substantially circular cross-section. In this regard, in certain variants of this embodiment, the powder composition may include from about 100 micrograms to about 3200 micrograms of apomorphine or a pharmaceutically acceptable salt or ester thereof (based on the weight of the hydrochloride salt) and a carrier material. Preferably, the powder composition comprises from about 100 micrograms to about 1600 micrograms of said apomorphine, more preferably, about 200 micrograms to about 1600 micrograms of said apomorphine, more preferably, about 300 micrograms to about 1200 micrograms of said apomorphine, more preferably about 400 to about 1200 micrograms of said apomorphine. Most preferably, doses are provided in increments between 400 and 1200 micrograms, based upon the requirements and tolerance of the individual patients. For examples, doses may be provided of about 400, about 500, about 600, about 700, about 800, about 900, about 1000, about 100, and/or about 1200 micrograms of said apomorphine.

In other variants of this embodiment, the powder composition may include a carrier material and apomorphine or a pharmaceutically acceptable salt or ester thereof, the carrier material has an average particle size of from about 40 to about 70 microns, and at least 90 percent of said apomorphine has a particle size of 5 microns or less. In any event, the method further comprises directing the gas flow through the inlet port into the vortex chamber in a tangential direction; directing the gas flow through the vortex chamber so as to aerosolize the powder composition; and directing the gas flow with the powder composition out of the vortex chamber in an axial direction through an exit port, wherein a velocity of the gas flow at a distance of 300 mm outside of the exit port is less than a velocity of the gas flow at the inlet port.

In accordance with another embodiment of the present invention, the method comprises entraining a powdered composition including agglomerated particles in a gas flow upstream from an inlet port of a vortex chamber. In certain variants of this embodiment, the agglomerated particles include from about 100 micrograms to about 3200 micrograms of apomorphine or a pharmaceutically acceptable salt or ester thereof (based on the weight of the hydrochloride salt) and a carrier material. Preferably, the agglomerated particles comprise from about 100 micrograms to about 1600 micrograms of said apomorphine, more preferably, about 200 micrograms to about 1600 micrograms of said apomorphine, more preferably, about 300 micrograms to about 1200 micrograms of said apomorphine, more preferably about 400 to about 1200 micrograms of said apomorphine. Most preferably, doses are provided in increments between 400 and 1200 micrograms, based upon the requirements and tolerance of the individual patients. For examples, doses may be provided of about 400, about 500, about 600, about 700, about 800, about 900, about 1000, about 100, and/or about 1200 micrograms of said apomorphine.

In other variants of this embodiment, the agglomerated particles include a carrier material and apomorphine or a pharmaceutically acceptable salt or ester thereof, the carrier material has an average particle size of from about 40 to about 70 microns, and at least 90 percent of said apomorphine has a particle size of 5 microns or less. In either case, the method further comprises directing the gas flow through the inlet port into the vortex chamber; depositing the agglomerated particles onto one or more walls of the vortex chamber; applying, via the gas flow through the vortex chamber, a shear to the deposited agglomerated particles to deagglomerate said particles, and directing the gas flow, including the deagglomerated particles, out of the vortex chamber, wherein a velocity of the gas flow at a distance of 300 mm outside of the exit port is less than a velocity of the gas flow at the inlet port.

In accordance with another embodiment of the present invention, the method comprises entraining agglomerated particles in a gas flow. The agglomerated particles include a carrier material having an average particle size of from about 40 microns to about 70 microns and from about 100 to about 3200 micrograms apomorphine or a pharmaceutically acceptable salt or ester thereof (based on the weight of the hydrochloride salt). Preferably, the agglomerated particles comprise a dose of from about 100 micrograms to about 1600 micrograms of said apomorphine, more preferably, about 200 micrograms to about 1600 micrograms of said apomorphine, more preferably, about 300 micrograms to about 1200 micrograms of said apomorphine, more preferably about 400 to about 1200 micrograms of said apomorphine. Most preferably, doses are provided in increments between 400 and 1200 micrograms, based upon the requirements and tolerance of the individual patients. For examples, doses may be provided of about 400, about 500, about 600, about 700, about 800, about 900, about 1000, about 100, and/or about 1200 micrograms of said apomorphine. Preferably, at least 90% of said apomorphine has a particle size of 5 microns or less. The method further comprises depositing the agglomerated particles onto one or more surfaces; and applying, via the gas flow, a shear to the deposited agglomerated particles to deagglomerate said particles.

In accordance with another embodiment of the present invention, the method comprises generating an air flow through an inlet port of a chamber, the air flow having entrained therein a composition. In certain variants of this embodiment, the composition comprises from about 100 micrograms to about 3200 micrograms of apomorphine or a pharmaceutically acceptable salt or ester thereof (based on the weight of the hydrochloride salt) and a carrier material. Preferably, the agglomerated particles comprise a dose of from about 100 micrograms to about 1600 micrograms of said apomorphine, more preferably, about 200 micrograms to about 1600 micrograms of said apomorphine, more preferably, about 300 micrograms to about 1200 micrograms of said apomorphine, more preferably about 400 to about 1200 micrograms of said apomorphine. Most preferably, doses are provided in increments between 400 and 1200 micrograms, based upon the requirements and tolerance of the individual patients. For examples, doses may be provided of about 400, about 500, about 600, about 700, about 800, about 900, about 1000, about 100, and/or about 1200 micrograms of said apomorphine. In other variants of this embodiment, the composition includes a carrier material and apomorphine or a pharmaceutically acceptable salt or ester thereof, the carrier material has an average particle size of from about 40 to about 70 microns, and at least 90 percent of said apomorphine has a particle size of 5 microns or less. The method further comprises directing the air flow through the chamber. The chamber has an axis and a wall curved about the axis and the air flow rotates about the axis. The method further directs the air flow through an exit port of the chamber, wherein a direction of the air flow through the inlet port is tangential to the wall, and a direction of the air flow through the exit port is parallel to the axis, and wherein a cross-sectional area of the air flow through the chamber is in a plane normal to the air flow and decreases with increasing distance from the inlet port.

In accordance with another embodiment of the present invention, a method of treating sexual dysfunction is provided, comprising inhaling a dose of a powder composition, the powder composition comprising agglomerated particles which include from about 100 to about 3200 micrograms of apomorphine or a pharmaceutically acceptable salt or ester thereof (based on the weight of the hydrochloride salt) and a carrier material. Preferably, the agglomerated particles comprise a dose of from about 100 micrograms to about 1600 micrograms of said apomorphine, more preferably, about 200 micrograms to about 1600 micrograms of said apomorphine, more preferably, about 300 micrograms to about 1200 micrograms of said apomorphine, more preferably about 400 to about 1200 micrograms of said apomorphine. Most preferably, doses are provided in increments between 400 and 1200 micrograms, based upon the requirements and tolerance of the individual patients. For examples, doses may be provided of about 400, about 500, about 600, about 700, about 800, about 900, about 1000, about 100, and/or about 1200 micrograms of said apomorphine. The step of inhaling includes entraining the agglomerated particles in a gas flow upstream from an inlet port of a vortex chamber, directing the gas flow through the inlet port into the vortex chamber; depositing the agglomerated particles onto one or more walls of the vortex chamber; applying, via the gas flow through the vortex chamber, a shear to the deposited agglomerated particles to deagglomerate said particles, and directing the gas flow, including the deagglomerated particles, out of the vortex chamber to provide an ultrafine particle fraction, when measured by an Andersen Cascade Impactor, United States Pharmacopeia 26, Chapter 601 Apparatus 3 (2003), of at least about 70%.

In accordance with another embodiment of the present invention, a method of treating sexual dysfunction is provided, comprising inhaling a dose of a powder composition, the powder composition comprising from about 100 to about 3200 micrograms of apomorphine or a pharmaceutically acceptable salt or ester thereof (based on the weight of the hydrochloride salt). Preferably, the powder composition also includes a carrier. Preferably, the dose comprises from about 100 micrograms to about 1600 micrograms of said apomorphine, more preferably, about 200 micrograms to about 1600 micrograms of said apomorphine, more preferably, about 300 micrograms to about 1200 micrograms of said apomorphine, more preferably about 400 to about 1200 micrograms of said apomorphine. Most preferably, doses are provided in increments between 400 and 1200 micrograms, based upon the requirements and tolerance of the individual patients. For examples, doses may be provided of about 400, about 500, about 600, about 700, about 800, about 900, about 1000, about 100, and/or about 1200 micrograms of said apomorphine. In one variant of this embodiment, the step of inhaling comprises inhaling a dose having an ultrafine particle fraction, when measured by an Andersen Cascade Impactor, United States Pharmacopeia 26, Chapter 601 Apparatus 3 (2003), of at least about 70%. In another variant of this embodiment, the step of inhaling comprises inhaling a dose having a fine particle fraction, when measured by an Andersen Cascade Impactor, United States Pharmacopeia 26, Chapter Apparatus 3 (2003), of at least about 80%.

In other aspects, the present invention is directed to inhalers for producing an inhalable aerosol of a powdered apomorphine composition.

In accordance with these embodiments, an inhaler for producing an inhalable aerosol of a powdered apomorphine composition comprises: an aerosolizing device including a substantially tangential inlet port and a substantially axial exit port, one or more sealed blisters containing apomorphine or a pharmaceutically acceptable salt or ester thereof, and an input for removably receiving one of the blisters. The inhaler, upon actuation, couples the tangential inlet port with the powder composition in the received blister.

In certain variants of this embodiment, each blister contains a powder composition including a carrier material and from about 100 micrograms to about 3200 micrograms of apomorphine or a pharmaceutically acceptable salt or ester thereof (based on the weight of the hydrochloride salt), as described above. In other variants, each blister contains a powder composition including a carrier material and apomorphine or a pharmaceutically acceptable salt or ester thereof, the carrier material has an average particle size of from about 40 to about 70 microns, and at least 90 percent of said apomorphine has a particle size of 5 microns or less, as described above.

Although certain of the compositions, methods or treatment, inhalers, blisters, methods for inhaling, and doses have been described above as including a carrier material having a preferred average particle size of from about 40 microns to about 70 microns, it should be appreciated that in accordance with other embodiments, the carrier material in these compositions, methods or treatment, inhalers, blisters, methods for inhaling, and doses can have other average particle size ranges, for example, from about 10 microns to about 1000 microns, from about 10 microns to about 70 microns, or from about 20 microns to about 120 microns.

In accordance with additional aspects of the above-referenced embodiments, a dose includes from about 400 to about 800 micrograms of apomorphine hydrochloride, and the dose provides, in vivo, a mean Cₘₐₓ of from about 0.7 ng/ml to about 2 ng/ml. Preferably, the dose provides, in vivo, a mean plasma level of said apomorphine at seventy minutes after administration of from about 0.2 ng/ml to about 0.6 ng/ml.

With regard to the aerosolizing device, in certain variants of this embodiment, the aerosolizing device is in the form a vortex chamber of substantially circular cross-section having a substantially tangential inlet port and a substantially axial exit port, wherein the ratio of the diameter of the vortex chamber to the diameter of the exit port is between 4 and 12.

In other variants, the aerosolizing device is in the form of a vortex chamber of substantially circular cross-section having a substantially tangential inlet port, wherein the inlet port has an outer wall which defines the maximum extent of the inlet port in the radially outward direction of the vortex chamber. The extent of the outer wall in the axial direction of the vortex chamber is substantially equal to the maximum extent of the inlet port in the axial direction of the vortex chamber, and the outer wall is substantially parallel with a wall of the vortex chamber.

In other variants, the aerosolizing device is in the form of a vortex chamber of substantially circular cross-section having a substantially tangential inlet port. An exit port is spaced from the inlet port in an axial direction. A bottom surface defines the furthest extent of the vortex chamber from the exit port in the axial direction, and the bottom surface further defines the furthest axial extent of the inlet port from the exit port.

In other variants, the aerosolizing device is in the form of a vortex chamber of substantially circular cross-section having a substantially tangential inlet port and an inlet conduit arranged to supply a powdered composition entrained in a gas flow to the inlet port, in use, wherein the cross-sectional area of the inlet conduit decreases towards the vortex chamber. The inlet conduit is, upon actuation of the inhaler, coupled to the powder composition in the received blister.

In other variants, the aerosolizing device is in the form of a vortex chamber of substantially circular cross-section having a substantially tangential inlet port and an arcuate inlet conduit arranged to supply a powdered composition entrained in a gas flow to the inlet port, in use. The inlet conduit is, upon actuation of the inhaler, coupled to the powder composition in the received blister.

In other variants, the aerosolizing device is in the form of a vortex chamber having an axis and being defined, at least in part, by a wall which forms a curve about the axis. The vortex chamber has a cross-sectional area in a plane bounded by the axis, and the plane extends in one direction radially from the axis at a given angular position (θ) about the axis. The vortex chamber has a substantially tangential inlet port and a substantially axial exit port, and said cross-sectional area of the vortex chamber decreases with increasing angular position (θ) in the direction, in use, of gas flow between the inlet port and the exit port.

In other variants, the aerosolizing device is in the form of a vortex chamber having an axis and being defined, at least in part, by a wall which forms a curve about the axis. The vortex chamber has a substantially tangential inlet port and a substantially axial exit port. The vortex chamber is further defined by a base, and the distance (d) between the base and a plane which is normal to the axis and is located on the opposite side of the base to the exit port increases with radial position (r) relative to the axis.

In other variants, the aerosolizing device includes a chamber defined by a top wall, a bottom wall, and a lateral wall, the lateral wall being curved about an axis which intersects the top wall and the bottom wall. The chamber encloses a cross-sectional area defined by the axis, the top wall, the bottom wall and the lateral wall, and the chamber has an inlet port and an outlet port. The inlet port is tangent to the lateral wall, the outlet port is co-axial with the axis, and the cross-sectional area decreases with increasing angular position from the inlet port in a direction of a gas flow through the inlet port.

In still other variants, the aerosolizing device a chamber including a wall, a base, an inlet port and an exit port. The chamber has an axis that is co-axial with the exit port and intersects the base. The wall is curved about the base, the inlet port is tangential to the wall, and a height between the base and a plane normal to the axis at the exit port decreases as a radial position from the axis to the inlet port increases.

### BRIEF DESCRIPTION OF THE DRAWINGS

FIG. 1 shows an inhaler and blister in accordance with an embodiment of the present invention.

FIG. 2 is a top cross-section of a vortex nozzle 1.

FIG. 3 is a side cross-section of a vortex chamber in accordance with an embodiment of the invention.

FIG. 4 is a sectional view along line B-B of the vortex chamber of FIG. 3.

FIG. 5(a) is a side view of a vortex chamber with a round inlet port.

FIG. 5(b) is a sectional view along line D-D of the vortex chamber of FIG. 5(a).

FIG. 6(a) is a side view of a vortex chamber with a rectangular inlet port.

FIG. 6(b) is a sectional view along line E-E of the vortex chamber of FIG. 6(a).

FIG. 7 shows a vortex chamber with an arcuate inlet conduit.

FIGS. 8 to 11 show detail of embodiments of the exit port of the inhaler in accordance with the invention.

FIG. 12 illustrates an asymmetric vortex chamber in accordance with an embodiment of the invention.

FIG. 13 is a sectional view of a vortex chamber in accordance with another asymmetric inhaler in accordance with an embodiment of the invention.

FIG. 14 is a perspective view of a vortex chamber according to FIG. 13.

FIG. 15 is a sectional view of the vortex chamber of FIG. 14.

FIG. 16 is a perspective view of a detail of the vortex chamber of FIGS. 14 and 15;

FIG. 17 is a plan view of the detail of FIG. 16.

FIG. 18 is a plan view of a variation of the detail of FIG. 17.

FIGS. 19 to 21 show variations of the interface between the wall and the base of a vortex chamber according to the embodiments of FIGS. 13-18.

FIGS. 22(A-B) illustrates the particle size distribution of the lactose of Example 1.

FIGS. 23(A-B) illustrate the particle size distribution of the micronized apomorphine of Example 2.

FIG. 24 shows stability data for the 200 microgram apomorphine-lactose formulation of Examples 2(a) and 3.

FIG. 25 shows a perspective view of the prototype inhaler used to perform inhalation testing in accordance with Example 4.

FIG. 26 shows the inhaler of FIG. 25 with its cover removed to show the breath actuation mechanism and vortex nozzle.

FIG. 27 is a cross-section view through the vortex nozzle taken along line AA in FIG. 26.

FIG. 28A is a cross-section view taken along line BB in FIG. 26 showing the nozzle valve in the closed position.

FIG. 28B is a cross-section view taken along line BB in FIG. 26 showing the nozzle valve in the open position.

FIGS. 29(A) and 29(B) illustrate the results of tests performed on the apomorphine-lactose formulation of Examples 2 and 3.

FIG. 30 illustrate the particle size distribution of the micronized Leucine of Example 10.

FIG. 31 illustrates the quality of erection by treatment group for the patients of Example 14.

FIG. 32 illustrates the response rate by treatment group for the patients of Example 14.

FIG. 33 illustrates the onset and duration of effect for the group of patients treated with Placebo in Example 14.

FIG. 34 illustrates the onset and duration of effect for the group of patients treated with 200 μg of apomorphine in Example 14

FIG. 35 illustrates the onset and duration of effect for the group of patients treated with 400 μg apomorphine in Example 14.

FIG. 36 illustrates the onset and duration of effect for the group of patients treated with 800 μg apomorphine in Example 14.

FIG. 37 shows a comparison of the blood levels at 70 minutes after dosing (T₇₀) for each patient for the 400 microgram dose and the 800 microgram dose, and additionally shows the known mean Cₘₐₓ of 2 mg, 4 mg, and 5 mg Uprima™ sublingual tablets.

FIG. 38 illustrates the amount (in micrograms) in drug that was delivered to each of the 11 components of an ACI in Example 17.

FIG. 39 illustrates the amount (in micrograms) in drug that was delivered to each of the 11 components of an ACI in Example 18.

### DETAILED DESCRIPTION OF THE PREFERRED EMBODIMENTS

The embodiments of the present invention are directed to inhalable formulations of apomorphine or its pharmaceutically acceptable salts or esters and methods for preparing the same, methods for treatment of sexual dysfunction using said formulations, inhalers including said formulations, and methods for using said inhalers.

The inhalable formulations in accordance with the present invention are preferably administered via a dry powder inhaler (DPI) formulations, but can also be administered via pressurized metered dose inhaler (pMDI) formulations, or even via a nebulized system.

In the context of the present invention, the dose (e.g., in micrograms) of apomorphine or its pharmaceutically acceptable salts or esters will be described based upon the weight of the hydrochloride salt (apomorphine hydrochloride). As such, a dose of 100 micrograms of “apomorphine or its pharmaceutically acceptable salts or esters” means 100 micrograms of apomorphine hydrochloride, or an equivalent amount of another salt, an ester, or of the base.

### Dry Powder Inhaler Formulations

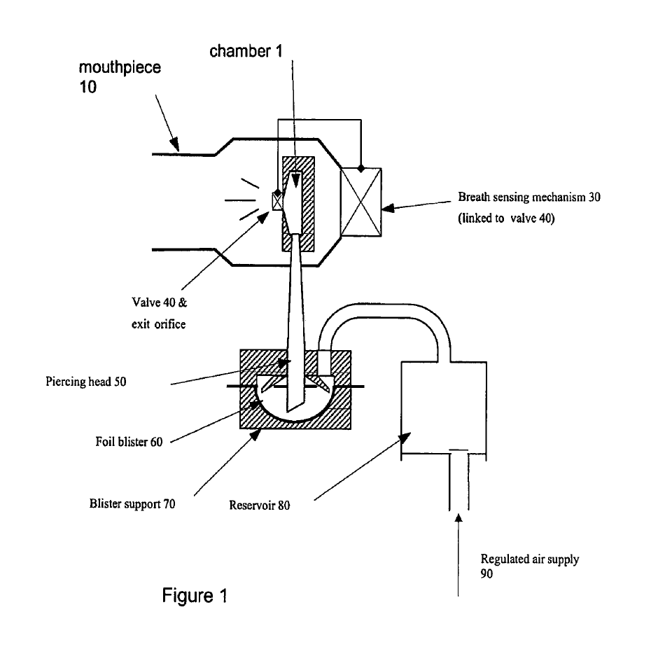

In a dry powder inhaler, the dose to be administered is stored in the form of a non-pressurized dry powder and, on actuation of the inhaler, the particles of the powder are inhaled by the patient. Dry powder inhalers can be “passive” devices in which the patient's breath is the only source of gas which provides a motive force in the device, or “active” devices in which a source of compressed gas or alternative energy source is used. Examples of “passive” dry powder inhaler devices include the Rotahaler and Diskhaler (Glaxo-Wellcome) and the Turbohaler (Astra-Draco). Particularly preferred “active” dry powder inhalers will be described in more detail below in connection with FIGS. 1-21, 25-29(b). It should be appreciated, however, that the compositions of the present invention can be administered with either passive or active inhaler devices.

“Actuation of the inhaler” refers to the process during which a dose of the powder is removed from its rest position in the inhaler (e.g., a blister, reservoir, or other container) usually by a patient inhaling. That step takes place after the powder (or container or blister containing the powder) has been loaded into the inhaler ready for use.

While it is clearly desirable for as large a proportion as possible of the particles of active material to be delivered to the deep lung, it is usually preferable for as little as possible of the other components to penetrate the deep lung. Therefore, powders generally include particles of an active material, and carrier particles for carrying the particles of active material.

As described in WO 01/82906, published Nov. 8, 2001, an additive material may also be provided in a dose which indicates to the patient that the dose has been administered. The additive material, referred to below as indicator material, may be present in the powder as formulated for the dry powder inhaler, or be present in a separate form, such as in a separate location within the inhaler such that the additive becomes entrained in the airflow generated on inhalation simultaneously or sequentially with the powder containing the active material.

In accordance with an embodiment of the present invention, an inhalable powder composition is provided which includes apomorphine or a pharmaceutically acceptable salt or ester thereof (thereinafter collectively “apomorphine”), in combination with a carrier material. An example of a suitable apomorphine ester is diisobutyryl apomorphine. Preferably, the apomorphine comprises apomorphine hydrochloride. In any event, the apomorphine is provided in an amount from about 100 micrograms to about 3200 micrograms per unit dose. Preferably, the apomorphine is provided in from about 100 micrograms to about 1600 micrograms per dose, more preferably, about 200 micrograms to about 1600 micrograms per dose, more preferably, about 300 micrograms to about 1200 micrograms per dose, more preferably about 400 to about 1200 micrograms of said apomorphine. Most preferably, doses are provided in increments between 400 and 1200 micrograms, based upon the requirements and tolerance of the individual patients. For examples, doses may be provided of about 400, about 500, about 600, about 700, about 800, about 900, about 1000, about 100, and/or about 1200 micrograms of said apomorphine.

These powder compositions, when inhaled, preferably exhibit a time to therapeutic effect of less than 15 minutes, preferably less than about 10 minutes, and most preferably less than about 9 minutes. It is further believed that these powder formulations, when inhaled, will have a therapeutic duration of about 1 to 1½ hours. Such a relatively short time period from administration through termination of therapeutic effect (about 1 hour to about 1¾ hours) is advantageous because apomorphine hydrochloride is known to have side effects such as drowsiness which may impair the patient from performing certain tasks, such as operating a motor vehicle or heavy equipment.

In certain embodiments of the present invention, each dose is stored in a “blister” of a blister pack. In this regard, apomorphine is susceptible to oxidation, and, as such, it is important to prevent (or substantially limit) oxidation of the apomorphine prior to administration. In accordance with the embodiments of the present invention which utilize blisters, exposure of the formulation to air prior to administration (and unacceptable oxidation of the apomorphine) is prevented by storing each dose in a sealed blister. Most preferably, oxidation is further prevented (or limited) by placing a plurality of blisters into a further sealed container, such as a sealed bag made, for example of a foil such as aluminum foil. The use of the sealed blisters (and optional sealed bags) eliminates any need to include anti-oxidants in the formulation.

For the effective administration by a dry powder inhaler of the particles of apomorphine material to the lung where they can be absorbed, the particle size characteristics of the powder are particularly important.

In particular, for the effective delivery of active material deep into the lung, the active particles should be small and well dispersed on actuation of the inhaler.

It is preferred for the powder to be such that a fine particle fraction of at least 35% is generated on actuation of the inhaler device. It is particularly preferred that the fine particle fraction be greater than or equal to 60%, more preferably at least 70%, and most preferably at least 80%.

Thus, in certain embodiments of the present invention also provide a powder for use in an inhaler device, the powder comprising apomorphine or a pharmaceutically acceptable salt or ester thereof in combination with a carrier material, the powder being such that it generates a fine particle fraction of at least 35%, preferably at least 45%, more preferably at least 50%, even more preferably at least 60%, even more preferably at least 70%, and most preferably at least 80%.

The term “fine particle dose” (FPD) is used herein to mean the total amount (e.g., in micrograms) of active material (in this case apomorphine or its pharmaceutically acceptable salts or esters) delivered by a device which has a diameter of not more than 5 μm. The term “ultrafine particle dose” (UFPD) is used herein to mean the total amount (e.g., in micrograms) of active material delivered by a device which has a diameter of not more than 3 μm. The total amount of active material delivered by a device (the delivered dose) is in general less than the amount of the active material that is metered in the device (the metered dose) or is present in a pre-metered dose within the device (the total dose). The term “fine particle fraction” (FPF) is used herein to mean that percentage of the total amount of active material delivered by a device which has a diameter of not more than 5 μm (i.e., FPF=100*FPD/delivered dose). The term “ultrafine particle fraction” is used herein to mean that percentage of the total amount of active material delivered by a device which has a diameter of not more than 3 μm. The term percent fine particle dose (% FPD) is used herein to mean the percentage of the total dose which is delivered with a diameter of not more than 5 μm (i.e., % FPD=100*FPD/total dose). The term percent ultrafine particle dose (% UFPD) is used herein to mean the percentage of the total dose which is delivered with a diameter of not more than 3 μm (i.e., % UFPD=100*UFPD/total dose).

Fine particle fractions, Ultrafine particle fractions, and Fine particles doses referred to herein in relation to powders can be measured using a sample of the powder fired from a dry powder inhaler into a Multi Stage Liquid Impinger (MSLI) (United States Pharmacopeia (U.S.P) 26, chapter 601, Apparatus 4, (2003) Apparatus C, European Pharmacopoeia, Method 5.2.9.18, Supplement 2000) or Anderson Cascade Impactor (ACI)(U.S.P. 26, chapter 601, Apparatus 3 (2003)). The powder is preferably such that a fine particle fraction of at least 35%, preferably at least 45%, more preferably at least about 50%, even more preferably at least 60%, even more preferably at least 70%, and most preferably at least 80%, is generated on actuation of the inhaler device.

Most preferably, the inhaler device is a high turbulence inhaler device, the arrangement being such that a fine particle fraction of at least 35%, preferably at least 50%, even more preferably at least 60%, even more preferably at least 70%, and most preferably at least 80% is generated on actuation of the inhaler device.

In accordance with another embodiment of the present invention, the dose of apomorphine or a pharmaceutically acceptable salt or ester thereof is defined in terms of the fine particle dose of the administered dose. The percentage of the apomorphine in the dose which will reach the lung (the % FPD) is dependent on the formulation used and on the inhaler used. As such, a 2000 microgram dose of apomorphine hydrochloride will deliver 700 micrograms of apomorphine to the lung of a patient if an % FPD of 35% is achieved, but deliver 1200 micrograms of apomorphine to the lung of a patient if an % FPD of 60% is achieved. As such, it is appropriate to define the dose of apomorphine in terms of the FPD of the formulation and inhaler used, as measured by a Multistage Liquid Impinger or an Anderson Cascade Impactor.

As such, in accordance with another embodiment of the present invention, a method for treating sexual dysfunction via inhalation is provided which comprises inhaling a dose of a powder composition into the lungs of a patient, the dose of the powder composition delivering, in vitro, a fine particle dose of from about 100 micrograms to about 1600 micrograms of apomorphine or pharmaceutically acceptable salt(s) or ester(s) thereof (based on the weight of the hydrochloride salt), when measured by a Multistage Liquid Impinger, United States Pharmacopeia 26, Chapter 601 Apparatus 4 (2003). Preferably, the dose delivers, in vitro, a fine particle dose from about 200 micrograms to about 1000 micrograms of said apomorphine, more preferably, about 200 micrograms to about 800 micrograms of said apomorphine, more preferably, about 200 micrograms to about 600 micrograms of said apomorphine, and most preferably about 200 to about 400 micrograms of said apomorphine when measured by a Multistage Liquid Impinger, United States Pharmacopeia 26, Chapter 601 Apparatus 4 (2003). The dose, defined in the manner above in connection with the Multistage Liquid Impinger, can similarly be used in connection with the blisters, inhalers, and compositions described herein.

In addition to the fine particle fraction, another parameter of interest is the ultrafine particle fraction defined above. Although particles having a diameter of less than 5 microns (corresponding to the FPF) are suitable for local delivery to the lungs, it is believed that for systemic delivery, even finer particles are needed, because the drug must reach the alveoli to be absorbed into the bloodstream. As such, it is particularly preferred that the formulations and devices in accordance with the present invention be sufficient to provide an ultrafine particle fraction of at least about 50%, more preferably at least about 60% and most preferably at least about 70%.

A “high turbulence inhaler device” is to be understood as meaning an inhaler device which is configured to generate relatively high turbulence within the device and/or a relatively high incidence of impaction of powder upon internal surfaces and/or obstructions within the device, whereby efficient de-agglomeration of agglomerated powder particles occurs in use of the device.

As noted above, in addition to the active material (and an indicator material if present), the powder preferably includes carrier material in the form of particles for carrying the particles of active material. The carrier particles may be composed of any pharmacologically inert material or combination of materials which is acceptable for inhalation.

Advantageously, the carrier particles are composed of one or more crystalline sugars; the carrier particles may be composed of one or more sugar alcohols or polyols. Preferably, the carrier particles are particles of dextrose or lactose, especially lactose.

Preferably, at least 90% by weight of the active material has a particle size of not more than 10 μm, most preferably not more than 5 μm. The particles therefore give a good suspension on actuation of the inhaler.

In embodiments of the present invention which utilize conventional inhalers, such as the Rotohaler and Diskhaler described above, the particle size of the carrier particles may range from about 10 microns to about 1000 microns. In certain of these embodiments, the particle size of the carrier particles may range from about 20 microns to about 120 microns. In certain other ones of these embodiments, the size of at least 90% by weight of the carrier particles is less than 1000 μm and preferably lies between 60 μm and 1000 μm. The relatively large size of these carrier particles gives good flow and entrainment characteristics.

In these embodiments, the powder may also contain fine particles of an excipient material, which may for example be a material such as one of those mentioned above as being suitable for use as a carrier material, especially a crystalline sugar such as dextrose or lactose. The fine excipient material may be of the same or a different material from the carrier particles, where both are present. The particle size of the fine excipient material will generally not exceed 30 μm, and preferably does not exceed 20 μm. In some circumstances, for example, where any carrier particles and/or any fine excipient material present is of a material itself capable of inducing a sensation in the oropharyngeal region, the carrier particles and/or the fine excipient material can constitute the indicator material. For example, the carrier particles and/or any fine particle excipient may comprise mannitol.

The powders may also be formulated with additional excipients to aid delivery and release. For example, powder may be formulated with relatively large carrier particles which aid the flow properties of the powder. Large carrier particles are known, and include lactose particles having a mass medium aerodynamic diameter of greater than 90 microns. Alternatively, hydrophobic microparticles may be dispersed within a carrier material. For example, the hydrophobic microparticles may be dispersed within a polysaccharide matrix, with the overall composition formulated as microparticles for direct delivery to the lung. The polysaccharide acts as a further barrier to the immediate release of the active agent. This may further aid the controlled release process. An example of a suitable polysaccharide is xanthan gum. Preferred hydrophobic materials include solid state fatty acids such as oleic acid, lauric acid, palmitic acid, stearic acid, erucic acid, behenic acid, or derivatives (such as esters and salts) thereof. Specific examples of such materials include phosphatidylcholines, phosphatidylglycerols and other examples of natural and synthetic lung surfactants. Particularly preferred materials include metal stearates, in particular magnesium stearate, which has been approved for delivery via the lung.

In some circumstances, the powder for inhalation may be prepared by mixing the components of the powder together. For example, the powder may be prepared by mixing together particles of active material and lactose.

The dry powder inhaler devices in which the powder compositions of the present invention will commonly be used include “single dose” devices, for example the Rotahaler, the Spinhaler and the Diskhaler in which individual doses of the powder composition are introduced into the device in, for example, single dose capsules or blisters, and also multiple dose devices, for example the Turbohaler in which, on actuation of the inhaler, one dose of the powder is removed from a reservoir of the powder material contained in the device.

As already mentioned, in the case of certain powders, a form of device that promotes high turbulence offers advantages in that a higher fine particle fraction will be obtainable than in the use of other forms of device. Such devices include, for example, the Turbohaler™ or Novolizer™, and may be devices of the kind in which generation of an aerosolized cloud of powder is driven by inhalation of the patient or of the kind having a dispersal device for generating or assisting in generation of the aerosolized cloud of powder for inhalation.

Where present, the amount of carrier particles will generally be up to 95%, for example, up to 90%, advantageously up to 80% and preferably up to 50% by weight based on the total weight of the powder. The amount of any fine excipient material, if present, may be up to 50% and advantageously up to 30%, especially up to 20%, by weight, based on the total weight of the powder.

In contrast to the particle sizes described above, in embodiments of the present invention which utilize an inhaler of the type described below in connection with FIGS. 1-21, 25-28, the carrier particles are preferably between 10 and 70 microns, and more preferably between 40 and 70 microns in diameter. Such a particle size can be achieved for example, by sieving the excipient through screens of 45 microns and 63 microns, thereby excluding particles that pass through the 45 micron screen, and excluding particles that do not pass through the 63 micron screen. Most preferably, the excipient is lactose. Preferably, at least 90% (percent), and most preferably at least 99%, of the apomorphine particles are 5 microns or less in diameter. As detailed below, such a formulation, when administered via the preferred inhalers of FIGS. 1-21, 25-28, can provide a fine particle fraction in excess of about 80%, and an ultrafine particle fraction in excess of about 70%.

The formulations described herein may also include one or more force control additives (FCAs) in addition to the carrier and the apomorphine. The FCAs may be provided in an amount from about 0.1% to about 10% by weight, and preferably from about 0.15% to 5%, most preferably from about 0.5% to about 2%. In the context of the present invention, FCAs include, but are not limited to, anti-adherent materials. FCAs may include, for example, magnesium stearate, leucine, lecithin, and sodium stearyl fumarate, and are described more fully in U.S. Pat. No. 6,153,224, which is hereby incorporated by reference.

When the FCA is micronized leucine or lecithin, it is preferably provided in an amount from about 0.1% to about 10% by weight. Preferably, the FCA comprises from about 3% to about 7%, preferably about 5%, of micronized leucine. Preferably, at least 95% by weight of the micronized leucine has a particle diameter of less than 150 microns, preferably less than 100 microns, and most preferably less than 50 microns. Preferably, the mass median diameter of the micronized leucine is less than 10 microns.

If magnesium stearate or sodium stearyl fumarate is used as the FCA, it is preferably provided in an amount from about 0.05% to about 5%, preferably from about 0.15% to about 2%, most preferably from about 0.25 to about 0.5%.

Where reference is made to particle size of particles of the powder, it is to be understood, unless indicated to the contrary, that the particle size is the volume weighted particle size. The particle size may be calculated by a laser diffraction method. Where the particle also includes an indicator material on the surface of the particle, advantageously the particle size of the coated particles is also within the preferred size ranges indicated for the uncoated particles.

In certain embodiments of the present invention, the apomorphine formulation is a “carrier free” formulation, which includes only the apomorphine or its pharmaceutically acceptable salts or esters and one or more anti-adherents. Such carrier free formulations are described in WO 97/03649, the entire disclosure of which is hereby incorporated by reference. In accordance with these embodiments, the powder formulation includes apomorphine or a pharmaceutically acceptable salt or ester thereof and an additive material which includes an anti-adherent material. The powder includes at least 60% by weight of the apomorphine or a pharmaceutically acceptable salt or ester thereof based on the weight of the powder. Advantageously, the powder comprises at least 70%, more preferably at least 80% by weight of apomorphine or a pharmaceutically acceptable salt or ester thereof based on the weight of the powder. Most advantageously, the powder comprises at least 90%, more preferably at least 95%, more preferably at least 97%, by weight of apomorphine or a pharmaceutically acceptable salt or ester thereof based on the weight of the powder. It is believed that there are physiological benefits in introducing as little powder as possible to the lungs, in particular material other than the active ingredient to be administered to the patient. Therefore, the quantities in which the additive material is added are preferably as small as possible. The most preferred powder, therefore, would comprise more than 99% by weight of apomorphine or a pharmaceutically acceptable salt or ester thereof.

In the context of the present invention, the term anti-adherent material refers to those additive materials which will decrease the cohesion between the particles of the powder. Those materials will include those usually thought of as anti-adherent materials, for example leucine, as well as others, for example, lecithin, which are not generally thought of as being anti-adherent but may nonetheless have the effect of decreasing the cohesion between the particles of the powder. Other materials commonly added to powders for use in inhalers, for example lactose and various other carrier particle materials, are not anti-adherent materials per se but might be added to a powder in addition to a suitable anti-adherent material, for example leucine as indicated above.

Advantageously, in these “carrier free” formulations, at least 90% by weight of the particles of the powder have a particle size less than 63 microns, preferably less than 30 microns and more preferably less than 10 microns. As indicated above, the size of the apomorphine (or it pharmaceutically acceptable salts) particles of the powder should be within the range of about from 0.1 micron to 5 microns for effective delivery to the lower lung. Where the anti-adherent material is in the form of particles of material it may be advantageous for particles of the anti-adherent material to have a size outside the preferred range for delivery to the lower lung.

It is particularly advantageous for the anti-adherent material to comprise an amino acid. Amino acids have been found to give, when present as anti-adherent material, high respirable fraction of the active material and also good flow properties of the powder. A preferred amino acid is leucine, in particular L-leucine. Although the L-form of the amino acids is preferred, the D- and DL-forms may also be used. The anti-adherent material may comprise one or more of any of the following amino acids: leucine, isoleucine, lysine, valine, methionine, cysteine, phenylalanine. Advantageously, the powder includes at least 80%, preferably at least 90% by weight of apomorphine (or it pharmaceutically acceptable salts) based on the weight of the powder. Advantageously, the powder includes not more than 8%, more advantageously not more than 5% by weight of additive material based on the weight of the powder. As indicated above, in some cases it will be advantageous for the powder to contain about 1% by weight of additive material. The anti-adherent material may also (or alternatively) include magnesium stearate or colloidal silicon dioxide.

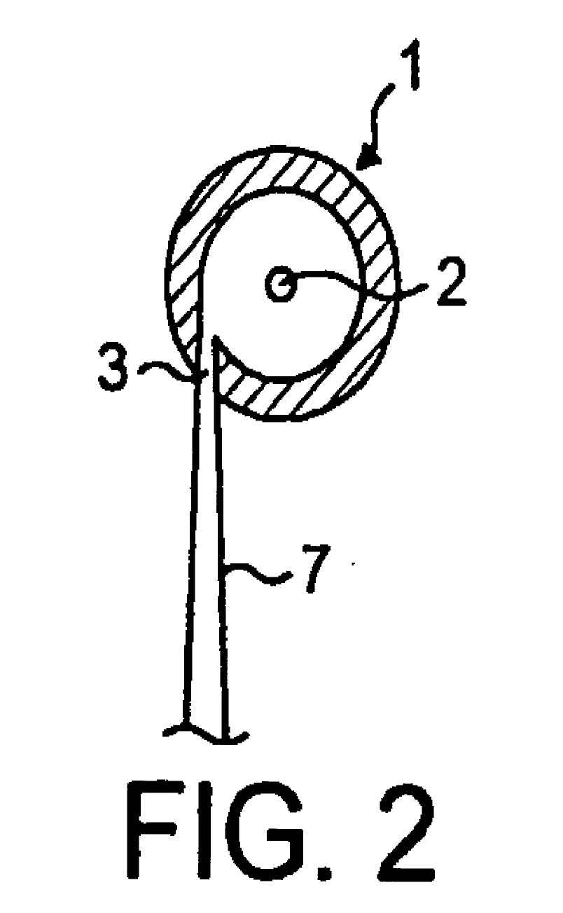

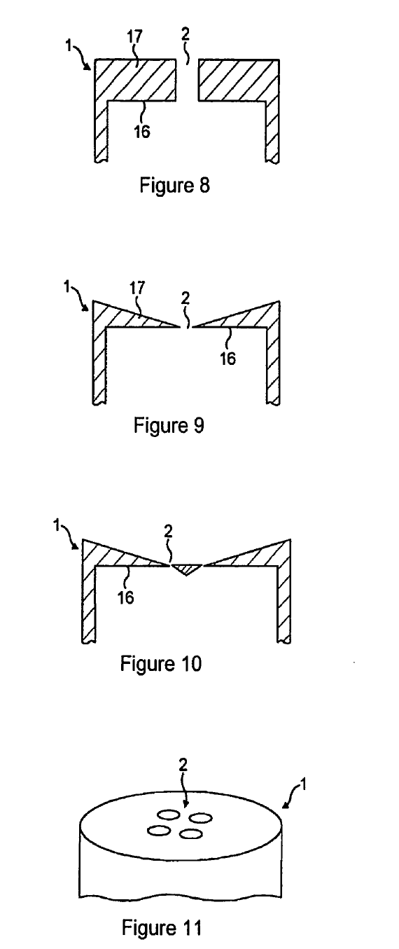

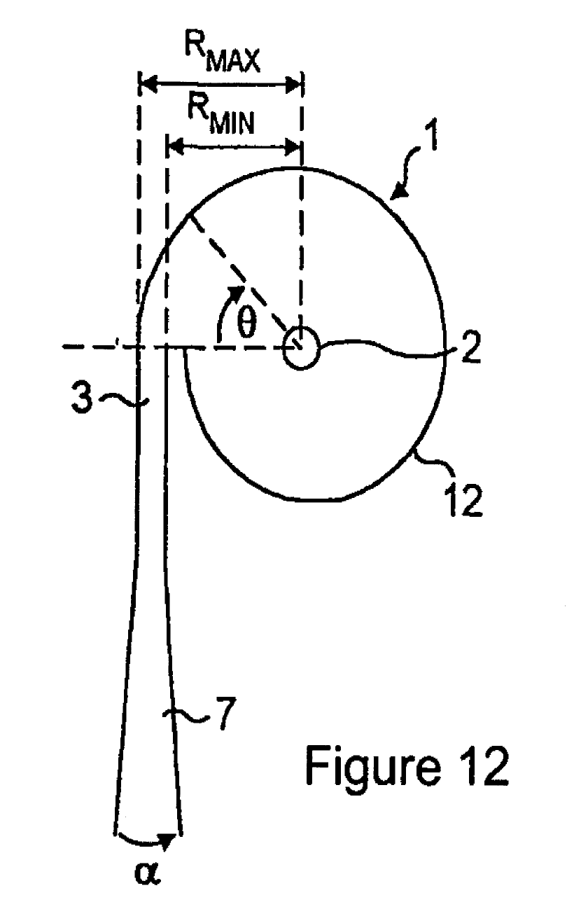

FIG. 1 shows schematically a preferred inhaler that can be used to deliver the powder formulations described above to a patient. Inhalers of this type are described in WO 02/089880 and WO 02/089881, both published on Nov. 14, 2002, the entire disclosures of which are hereby incorporated by reference. FIGS. 2-7 correspond to the inhalers described in WO 02/089880, FIGS. 12-21 correspond to the inhalers described in WO 02/089881, and FIGS. 8-11 show preferred exit port configurations that can be used in connection with any of these inhalers.

Referring to FIGS. 1 and 2, the inhaler comprises a nozzle 3000 including a vortex chamber 1 and having an exit port 2 and an inlet port 3 for generating an aerosol of the powder formulation. The vortex chamber 1 is located in a mouthpiece 10 through which the user inhales to use the inhaler. Air passages (not shown) may be defined between the vortex chamber 1 and the mouthpiece 10 so that the user is able to inhale air in addition to the powdered medicament.

The powder formulation is stored in a blister 60 defined by a support 70 and a pierceable foil lid 75. As shown, the support 70 has a cavity formed therein for holding the powder formulation. The open end of the cavity is sealed by the lid 75. An air inlet conduit 7 of the vortex chamber 1 terminates in a piercing head (or rod) 50 which pierces the pierceable foil lid 75. A reservoir 80 is connected to the blister 60 via a passage 78. A regulated air supply 90 charges the reservoir 80 with a gas (e.g., air, in this example) to a predetermined pressure (e.g. 1.5 bar). Preferably, the blister contains from 1 to 5 mg of powder formulation, preferably 1, 2 or 3 mg of powder formulation.

In certain embodiments, the support 70 is also made of foil. Such blisters are commonly referred to in the art as double-foil blisters. In other embodiments of the present invention, the support 70 is made of a polymer. It is believed that the foil support 70 provides greater protection against moisture and oxidation than the polymer support 70.

When the user inhales, a valve 40 is opened by a breath-actuated mechanism 30, forcing air from the pressurized air reservoir through the blister 60 where the powdered formulation is entrained in the air flow. The air flow transports the powder formulation to the vortex chamber 1, where a rotating vortex of powder formulation and air is created between the inlet port 3 and the outlet port 2. Rather than passing through the vortex chamber in a continuous manner, the powdered formulation entrained in the airflow enters the vortex chamber in a very short time (typically less than 0.3 seconds and preferably less than 20 milliseconds) and, in the case of a pure drug formulation (i.e., no carrier), a portion of the powder formulation sticks to the walls of the vortex chamber. This powder is subsequently aerosolised by the high shear forces present in the boundary layer adjacent to the powder. The action of the vortex deagglomerates the particles of powder formulation, or in the case of a formulation comprising a drug and a carrier, strips the drug from the carrier, so that an aerosol of powdered formulation exits the vortex chamber 1 via the exit port 2. The aerosol is inhaled by the user through the mouthpiece 10.

The vortex chamber 1 can be considered to perform two functions: deagglomeration, the breaking up of clusters of particles into individual, respirable particles; and filtration, preferentially allowing particles below a certain size to escape more easily from the exit port 2. Deagglomeration breaks up cohesive clusters of powdered formulation into respirable particles, and filtration increases the residence time of the clusters in the vortex chamber 1 to allow more time for them to be deagglomerated. Deagglomeration can be achieved by creating high shear forces due to velocity gradients in the airflow in the vortex chamber 1. The velocity gradients are highest in the boundary layer close to the walls of the vortex chamber.

As shown in more detail in FIG. 2, the vortex chamber 1 of FIGS. 2 through 7 is in the form of a substantially cylindrical chamber. The vortex chamber 1 has a frustoconical portion in the region of the exit port 2. The inlet port 3 is substantially tangential to the perimeter of the vortex chamber 1 and the exit port 2 is generally concentric with the axis of the vortex chamber 1. Thus, gas enters the vortex chamber 1 tangentially via the inlet port 3 and exits axially via the exit port 2. Between the inlet port 3 and the exit port 2 a vortex is created in which shear forces are generated to deagglomerate the particles of medicament. The length of the exit port 2 is preferably minimized to reduce the possibility of deposition of the drug on the walls of the exit port 2. In the embodiment shown, the vortex chamber 1 is machined from polyetheretherketone (PEEK), acrylic, or brass, although a wide range of alternative materials is possible.

The ratio of the diameter of the vortex chamber to the diameter of the exit port can be significant in maximising the fine particle fraction of the medicament aerosol which is expelled from the exit port. Thus, the ratio of the diameter of the vortex chamber to the diameter of the exit port may be between 4 and 12. It has been found that when the ratio is between 4 and 12 the proportion of particles of the powdered medicament with an effective diameter in the range 1 to 3 microns is maximised. For an enhanced fine particle fraction, the ratio is preferably greater than 5, most preferably greater than 6 and preferably less than 9, most preferably less than 8. In the preferred arrangement, the ratio is 7.1.

In certain embodiments of the invention, the diameter of the vortex chamber is between 2 and 12 mm. The diameter of the vortex chamber is preferably greater than 4 mm, most preferably at least 5 mm and preferably less than 8 mm, most preferably less than 6 mm. In the preferred embodiment, the diameter of the vortex chamber is 5 mm. In these embodiments, the height of the vortex chamber is generally between 1 and 8 mm. The height of the vortex chamber is preferably less than 4 mm, most preferably less than 2 mm. In the preferred embodiment, the height of the vortex chamber is 1.6 mm. In general, the vortex chamber is substantially cylindrical. However, the vortex chamber may take other forms. For example, the vortex chamber may be frustoconical. Where the diameter of the vortex chamber or the exit port is not constant along its length, the ratio of the largest diameter of the vortex chamber to the smallest diameter of the exit port should be within the range specified above. The aerosolising device comprises an exit port, for example as described above. The diameter of the exit port is generally between 0.5 and 2.5 mm. The diameter of the exit port is preferably greater than 0.6 mm and preferably less than 1.2 mm, most preferably less than 1.0 mm. In the preferred embodiment, the diameter of the exit port is 0.7 mm.

TABLE 1
Symmetrical Vortex chamber dimensions

| Dimension | | Preferred Value |
|---|---|---|
| D | Diameter of chamber | 5.0 mm |
| H | Height of chamber | 1.6 mm |
| h | Height of conical part of chamber | 0.0 mm |
| Dₑ | Diameter of exit port | 0.7 mm |
| t | Length of exit port | 0.3 mm |
| a | Height of inlet port | 1.1 mm |
| b | Width of inlet port | 0.5 mm |
| α | Taper angle of inlet conduit | 12° |

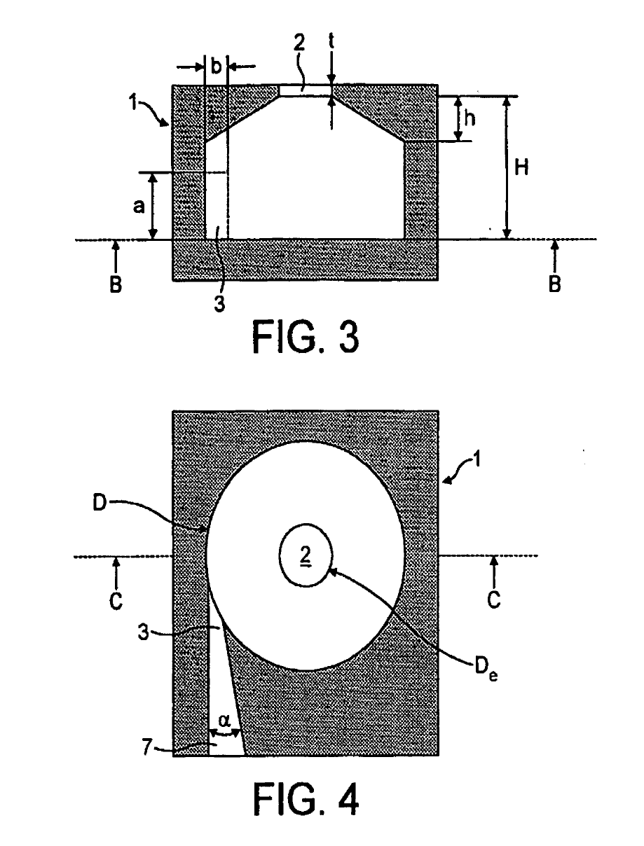

FIGS. 3 and 4 show the general form of the vortex chamber of the inhaler of FIG. 1. The geometry of the vortex chamber is defined by the dimensions listed in Table 1. The preferred values of these dimension are also listed in Table 1. It should be noted that the preferred value of the height h of the conical part of the chamber is 0 mm, because it has been found that the vortex chamber functions most effectively when the top of the chamber is flat.

As shown in Table 2, the proportion of the particles of medicament emitted in the aerosol having an effective particle diameter of less than 6.8 microns generated by the vortex chamber (the 6.8 micron particle fraction) depends on the ratio of the diameters of the chamber D and the exit port Dₑ. The normalised average 6.8 micron particle fraction is the emitted 6.8 micron particle fraction divided by the 6.8 micron particle fraction of the powdered medicament loaded into the inhaler. The medicament used was pure Intal™ sodium cromoglycate (Fisons UK).

TABLE 2
Relationship between emitted 6.8 micron particle fraction
and ratio of vortex chamber diameter to exit port diameter.

| Ratio | Average particle fraction that | Normalised average particle |
|---|---|---|
| D/Dₑ | is less than 6.8 μm | fraction that is less than 6.8 μm |
| 2.0 | 64.7% | 73.1% |
| 3.1 | 70.8% | 79.9% |
| 4.0 | 75.5% | 85.2% |
| 6.0 | 81.0% | 91.4% |
| 7.1 | 83.5% | 94.3% |
| 8.0 | 83.2% | 93.9% |
| 8.6 | 80.6% | 91.0% |

It will be seen from the above table that where the ratio of the diameters of the chamber and the exit port is 4 or more, the normalised 6.8 micron particle fraction is over 85%. Thus, the deagglomeration efficiency of the vortex chamber is significantly improved where the ratio is in this range. With the preferred ratio of 7.1, a normalised 6.8 micron particle fraction of 94.3% has been achieved.

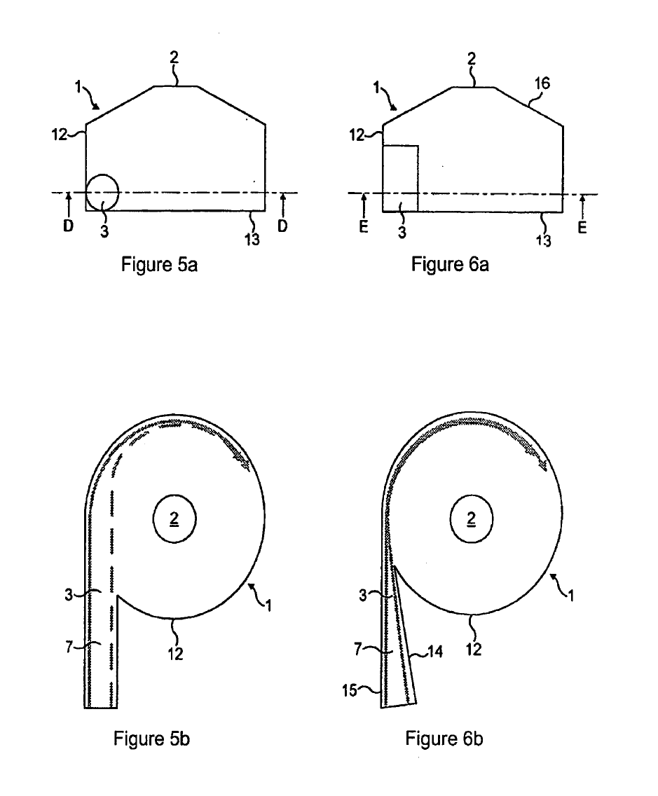

FIGS. 5a and 5b show a vortex chamber 1 in which the inlet port 3 has a circular cross-section. As represented by the solid arrow in FIG. 5b, a portion of the airflow entering the vortex chamber via the inlet port 3 follows the lateral wall 12 of the vortex chamber 1. The powder entrained in this airflow is therefore introduced directly into the airflow at the boundary layer adjacent the lateral wall 12 of the vortex chamber 1, where the velocity gradient in the radial direction is at a maximum. The maximal velocity gradient results in maximal shear forces on the agglomerated particles of the powder and thus maximum deagglomeration.

However, as represented by the dashed arrow in FIG. 5b, a portion of the airflow entering the vortex chamber via the inlet port 3 does not follow the chamber wall 12, but rather crosses the chamber 1 and meets the wall 12 at a point opposite the inlet port 3. At this point, there is increased turbulence, because the flow must make an abrupt change of direction. This turbulence disturbs the boundary layer adjacent the wall 12 of the chamber 1 and thereby reduces the effectiveness of the deagglomeration of the powder.

FIGS. 6a and 6b show a vortex chamber 1 in which the inlet port 3 has a rectangular cross-section. The rectangular cross-section maximises the length of the perimeter of the inlet port that is coincident with the wall 12 of the vortex chamber 1, such that the maximum air flow is introduced into the boundary layer of the vortex. Similarly, the rectangular cross-section maximises the width of the perimeter of the inlet port 3 that is coincident with the bottom surface 13 of the vortex chamber 1. In this way, deposition of powder in the vortex chamber 1 is prevented, because the vortex occupies the entire chamber 1.

In addition to having a rectangular cross-section, the inlet port 3 of FIGS. 6a and 6b is supplied by an inlet conduit 7 which tapers towards the vortex chamber 1. Thus, the inlet conduit 7 is defined by an inner wall 14 and an outer wall 15. The outer wall 15 is substantially tangential to the wall 12 of the vortex chamber 1. The spacing of the inner wall 14 from the outer wall 15 decreases towards the vortex chamber 1, so that the inner wall 14 urges the air flow into the vortex chamber 1 towards the boundary layer.

Furthermore, the decreasing cross-sectional area of the inlet conduit 7 causes the flow of velocity to increase, thereby reducing deposition of powder on the way to the vortex chamber 1.

As indicated by the arrows in FIG. 6b, all of the airflow entering the vortex chamber via the inlet port 3 follows the wall 12 of the vortex chamber 1. The powder entrained in this airflow is therefore introduced directly into the airflow at the boundary layer adjacent the wall 12 of the vortex chamber 1, and deagglomeration is maximised.

A further improvement can also be achieved if the upper surface 16 of the vortex chamber 1 is flat, as shown in FIGS. 8 to 10, rather than conical as shown in FIGS. 1, 3, 5 and 6. Thus, in this arrangement, the upper surface 16 of the vortex chamber 1 is substantially perpendicular to the wall 12 of the chamber 1, and to the axis of the vortex.

FIGS. 8 to 11 show various options for the exit port 2 of the vortex chamber 1. The characteristics of the exit plume of the aerosol are determined, at least in part, by the configuration of the exit port 2. For example, if the aerosol leaves an exit port 2 of 1 mm diameter at a flow rate of 2 litres/minute, the velocity at the exit port 2 will be approximately 40 m/s. This velocity can be reduced to a typical inhalation velocity of 2 m/s within a few centimetres of the chamber or nozzle by providing a strongly divergent aerosol plume.

In FIG. 8, the exit port 2 is a simple orifice defined through the upper wall 17 of the vortex chamber 1. However, the thickness of the upper wall 17 means that the exit port 2 has a length which is greater than its diameter. Thus, there is a risk of deposition in the exit port as the aerosol of powder exits. Furthermore, the tubular exit port tends to reduce the divergence of the exit plume. These problems are solved in the arrangement of FIG. 9 by tapering the upper wall 17 of the vortex chamber 1 towards the exit port 2 so that the exit port 2 is defined by a knife edge of negligible thickness. For an exit port 2 of diameter 1 mm, an exit port length of 2.3 mm gives a plume angle of 60°, whereas reducing this length to 0.3 mm increases the angle to 90°.

In FIG. 10, the exit port 11 is annular and is also defined by a knife edge. This arrangement produces an exit plume that slows down more quickly than a circular jet, because the annular exit port has a greater perimeter than a circular port of the same diameter and produces a jet that mixes more effectively with the surrounding static air. In FIG. 11, multiple orifices form the exit port 2 and produce a number of smaller plumes which break up and slow down in a shorter distance than a single large plume.

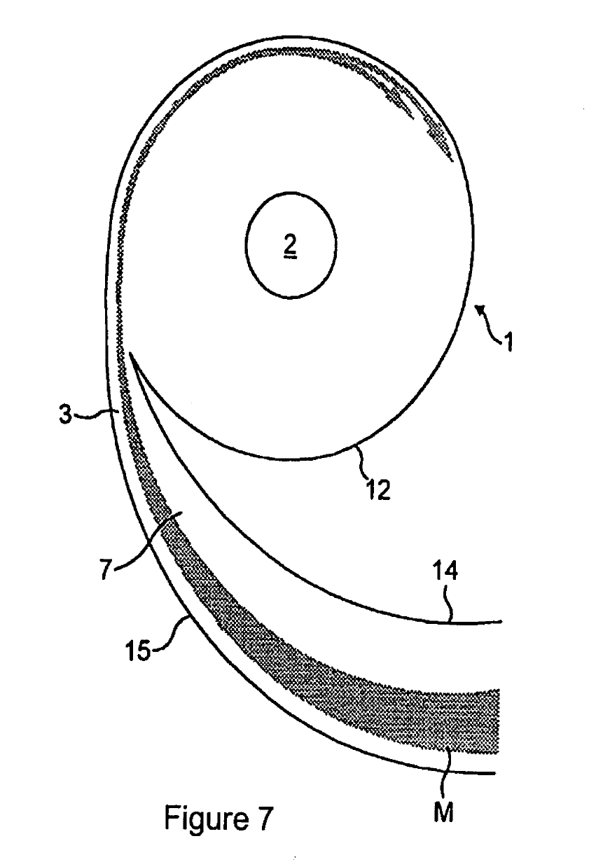

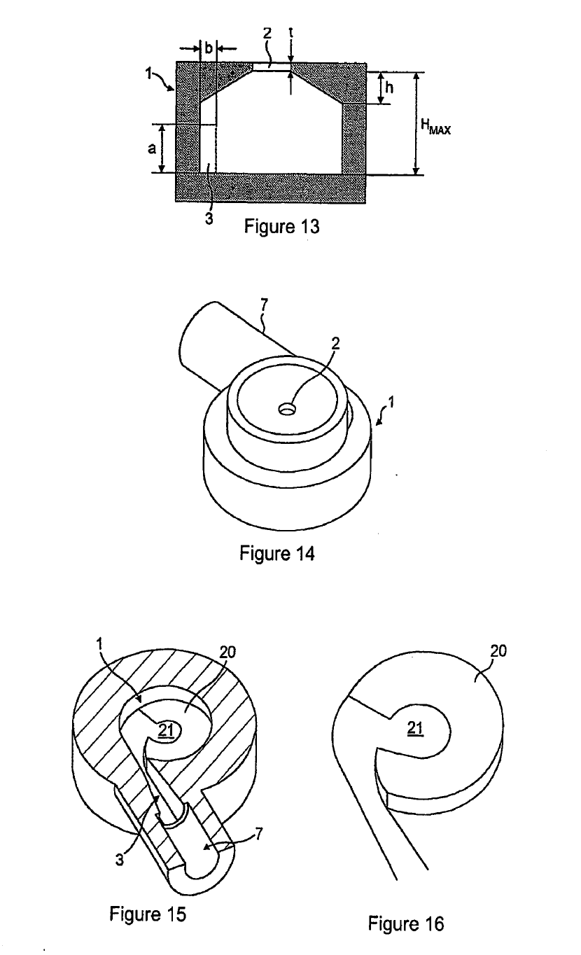

FIG. 7 shows an embodiment of the vortex chamber 1 in which the inlet conduit 7 is arcuate and tapers towards the vortex chamber 1. As shown by the arrows in FIG. 13, the arcuate inlet conduit 7 urges the entrained particles of powdered formulation towards the outer wall 15 of the inlet conduit 7. In this way, when the powder enters the vortex chamber 1 through the inlet port 3 the powder is introduced directly into the boundary layer next to the wall 12 of the vortex chamber 1, where shear forces are at a maximum. In this way, improved deagglomeration is achieved.

The inhaler in accordance with embodiments of the invention is able to generate a relatively slow moving aerosol with a high fine particle fraction. The inhaler is capable of providing complete and repeatable aerosolisation of a measured dose of powdered drug and of delivering the aerosolised dose into the patient's inspiratory flow at a velocity less than or equal to the velocity of the inspiratory flow, thereby reducing deposition by impaction in the patient's mouth. Furthermore, the efficient aerosolising system allows for a simple, small and low cost device, because the energy used to create the aerosol is small. The fluid energy required to create the aerosol can be defined as the integral over time of the pressure multiplied by the flow rate. This is typically less than 5 joules and can be as low as 3 joules.

FIGS. 12-21 show asymmetric inhalers in accordance with other embodiments of the present invention with similar components bearing identical reference numbers to the embodiments described above.

Initially, it should be noted that the difference between these embodiments and the embodiments described above with regard to FIGS. 1-11 is that, in the embodiments shown in FIGS. 12-21, the vortex chamber 1 has an asymmetric shape.

In the embodiment shown in FIG. 12, the wall 12 of the vortex chamber 1 is in the form of a spiral or scroll. The inlet port 3 is substantially tangential to the perimeter of the vortex chamber 1 and the exit port 2 is generally concentric with the axis of the vortex chamber 1. Thus, gas enters the vortex chamber 1 tangentially via the inlet port 3 and exits axially via the exit port 2. The radius R of the vortex chamber 1 measured from the centre of the exit port 2 decreases smoothly from a maximum radius Rₘₐₓ at the inlet port 3 to a minimum radius Rₘᵢₙ. Thus, the radius R at an angle θ from the position of the inlet port 3 is given by R=Rₘₐₓ(1−θk/2π), where k=(Rₘₐₓ−Rₘᵢₙ)/Rₘₐₓ. The effective radius of the vortex chamber 1 decreases as the air flow and entrained particles of medicament circulate around the chamber 1. In this way, the effective cross-sectional area of the vortex chamber 1 experienced by the air flow decreases, so that the air flow is accelerated and there is reduced deposition of the entrained particles of medicament. In addition, when the flow of air has gone through 2π radians (360°), the air flow is parallel to the incoming airflow through the inlet port 3, so that there is a reduction in the turbulence caused by the colliding flows.

Between the inlet port 3 and the exit port 2 a vortex is created in which shear forces are generated to deagglomerate the particles of the powdered formulation. As discussed above, the length of the exit port 2 is preferably as short as possible to reduce the possibility of deposition of the drug on the walls of the exit port 2. In the embodiment shown, the vortex chamber 1 is machined from PEEK, acrylic, or brass, although a wide range of alternative materials is possible. For manufacturing ease, the radius of the vortex chamber 1 may decrease in steps rather than smoothly.

FIG. 13 shows the general form of the vortex chamber of the inhaler of FIG. 12. The geometry of the vortex chamber is defined by the dimensions listed in Table 3. The preferred values of these dimension are also listed in Table 3. It should be noted that the preferred value of the height h of the conical part of the chamber is 0 mm, because it has been found that the vortex chamber functions most effectively when the top (roof 16) of the chamber is flat.

TABLE 3
Asymmetrical Vortex chamber dimensions

| Dimension | | Preferred Value |
|---|---|---|
| Rₘₐₓ | Maximum radius of chamber | 2.8 mm |
| Rₘᵢₙ | Minimum radius of chamber | 2.0 mm |
| Hₘₐₓ | Maximum height of chamber | 1.6 mm |
| h | Height of conical part of chamber | 0.0 mm |
| Dₑ | Diameter of exit port | 0.7 mm |
| t | Length of exit port | 0.3 mm |
| a | Height of inlet port | 1.1 mm |
| b | Width of inlet port | 0.5 mm |
| α | Taper angle of inlet conduit | 9°, then 2° |

The 6.8 micron particle fraction of the aerosol generated by the vortex chamber 1 according to FIG. 12 is improved relative to a circular vortex chamber of FIGS. 1-11.

FIGS. 14 to 18 show another asymmetric inhaler in accordance with the present invention in which the vortex chamber 1 includes a ramp 20 which reduces the height of the vortex chamber 1 from the bottom up with increasing angular displacement θ from the inlet port 3. A substantially circular region 21 in the centre of the vortex chamber 1 remains flat.

Various options for the cross-section of the ramp 20 are shown in FIGS. 19 to 21. As shown in FIG. 19, the cross-section of the ramp 20 may be a curve, such as a conic section. The value of the radius (or radii) of the curve may increase with increasing angular displacement θ about the axis of the vortex chamber 1.

Preferably, as shown in FIG. 20, the ramp 20 has a triangular cross-section, with an angle β between the base and the upper surface of the ramp 20. The angle β is a function of the angular displacement θ, such that β=q(θ−θ₁) where θ₁ and q are constants.

As shown in FIG. 21, the joints between the ramp 20 and the wall 12 of the vortex chamber and the ramp 20 and the base of the vortex chamber 1 are curved, for example with a fillet radius, to prevent unwanted deposition in this region.

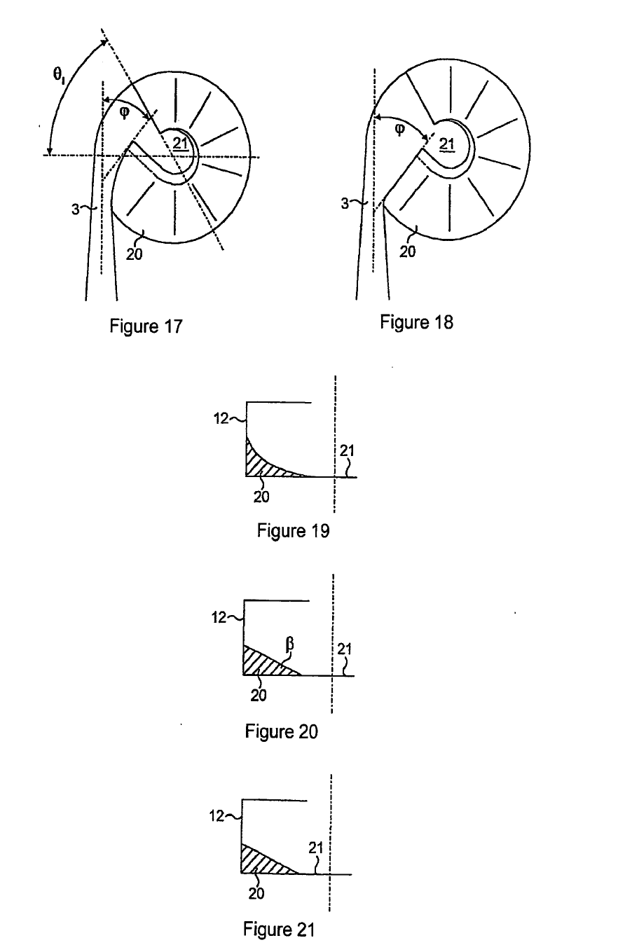

The vertical face (normal to the base) of the ramp 20 where the ramp meets the inlet 3 is likely to attract deposition because of the abrupt change in height. However, by arranging the profile of the face (looking axially) to form a smooth entry, as shown in FIG. 17, contiguous with the inner edge of the inlet 3 air travelling from the inlet scours the face and prevents powder build up.

In one arrangement the profile is a straight line at 40° (angle φ in FIG. 18) to the centre line of the inlet, joined to the inlet wall by a tangent curve. This profile follows the pattern of deposition that would be seen in a similar nozzle without a ramp.

In a preferred embodiment the profile is a curve moving radially inward as shown in FIG. 17. At one end it joins the inner wall of the inlet tangentially. At the other end it joins a continuation of the inner curve of the ramp at the point where the ramp meets the base.

### Pressurized Metered Dose Inhaler Formulations

Pressurized metered dose inhalers (pMDI) typically have two components: a canister component in which the drug particles (in this case apomorphine or its pharmaceutically acceptable salts or esters) are stored under pressure in a suspension or solution form and a receptacle component used to hold and actuate the canister. Typically, a canister will contain multiple doses of the formulation, although it is possible to have single dose canisters as well. The canister component typically includes a valved outlet from which the contents of the canister can be discharged. Aerosol medication is dispensed from the pMDI by applying a force on the canister component to push it into the receptacle component thereby opening the valved outlet and causing the medication particles to be conveyed from the valved outlet through the receptacle component and discharged from an outlet of the receptacle component. Upon discharge from the canister, the medication particles are “atomized” forming an aerosol. It is intended that the patient coordinate the discharge of aerosolized medication with his or her inhalation so that the medication particles are entrained in the patient's inspiratory flow and conveyed to the lungs. Typically, pMDIs use propellants to pressurize the contents of the canister and to propel the medication particles out of the outlet of the receptacle component. In pMDI inhalers, the formulation is provided in liquid form, and resides within the container along with the propellant. The propellant can take a variety of forms. For example, the propellant can comprise a compressed gas or a liquified gas. Suitable propellants include CFC (chlorofluorocarbon) propellants such as CFC 11 and CFC 12, as well as HFA (Hydrofluoroalkane) propellants such as HFA 134a and HFA 227. One or more propellants may be used in a given formulation.

In order to better coordinate actuation of the inhaler with inhalation, a breath actuated valve system may be used. Such systems are available, for example, from Baker Norton and 3M. To use such a device, the patient “primes” the device, and then the dose is automatically fired when the patient inhales.

In accordance with certain embodiments of the present invention, a pMDI formulation is used to deliver the apomorphine or its pharmaceutically acceptable salts or esters (thereinafter collectively “apomorphine”) to the lungs of the patient. The apomorphine is provided in an amount from about 100 micrograms to about 3200 micrograms per unit dose. Preferably, the dose comprises from about 100 micrograms to about 1600 micrograms of said apomorphine, more preferably, about 200 micrograms to about 1600 micrograms of said apomorphine, more preferably, about 300 micrograms to about 1200 micrograms of said apomorphine, more preferably about 400 to about 1200 micrograms of said apomorphine. Most preferably, doses are provided in increments between 400 and 1200 micrograms, based upon the requirements and tolerance of the individual patients. For examples, doses may be provided of about 400, about 500, about 600, about 700, about 800, about 900, about 1000, about 100, and/or about 1200 micrograms of said apomorphine.

In certain embodiments, the pMDI formulation is either a “suspension” type formulation or a “solution” type formulation, each using a liquified gas as the propellant. It is believed that the in vivo affect of pMDI formulations will be similar to those of the DPI formulations described above, in terms of time to therapeutic effect, and duration of therapeutic effect.

### Solution pMDI

Of pMDI technologies, solution pMDIs are believed to be the most appropriate for systemic lung delivery as they offer the finest mist, and can be more easily optimized through modifications to the device. Recently developed valves (e.g. available from Bespak) also offer payload increases over current systems, meaning that larger systemic doses can potentially be delivered in solution pMDIs than in suspension type pMDIs.

Solution pMDI techniques can be used to prepare formulations for delivery of apomorphine esters (for example, diisobutyryl apomorphine) with HFA propellants.

However, conventional solution pMDI techniques are not believed to be appropriate for the delivery of apomorphine or its pharmaceutically acceptable salts with HFA propellants. Specifically, apomorphine base is too unstable to be formulated using current approaches and apomorphine salts are too polar to be formulated as solutions in HFA propellants. For example, apomorphine HCl requires at least 50% ethanol for suitable or acceptable solubility in these systems, and such systems would neither be technologically acceptable or likely to be accepted by patients. Even with such a system, a solution concentration of <25 μg/dose is achieved, which is well below the effective doses described above in connection with Dry Powder Inhalers.

In the past, formulators sought to minimize the amount of water present in a pMDI solution because water was known to reduce the fine particle fraction of the formulation (e.g. as reported in WO 02/030499 to Chiesi) and/or to reduce the stability of the formulation (e.g., as reported in WO 01/89616 to Glaxo).

In accordance with an embodiment of the present invention, a pMDI solution including apomorphine or its pharmaceutically acceptable salts is surprisingly provided through the deliberate addition of water to the system. Specifically, it is believed that a suitable pMDI solution can be obtained by adding the apomorphine or its pharmaceutically acceptable salts to a propellant solution which includes from about 50% to about 98% w/w HFA134a (1,1,1,2-tetrafluoroethane) (and/or HFA 227 (1,1,1,2,3,3,3-heptafluoropropane)), from about 2% to about 10% w/w water, and from about 0% to about 47% w/w ethanol. Preferably, the water is provided in an amount from greater than 5% to about 10% w/w. With regard to ethanol, it is preferably provided in an amount from about 12% to about 40% w/w. Preferably, a 12ml solution would include about 170 milligrams of apomorphine hydrochloride in addition to the HFA134a, water and/or ethanol. A 3M coated (DUPONT 3200 200) canister can be used as the canister for the inhaler.

### Suspension pMDI

Suspension pMDIs can also be used to deliver apomorphine or its pharmaceutically acceptable salts to the lungs. However, suspension pMDIs have a number of disadvantages. For example, suspension pMDIs generally deliver lower doses than solution pMDIs and are prone to other issues related to suspensions e.g. dose inconsistencies, valve blockage, and suspension instabilities (e.g. settling). For these reasons, and others, suspension pMDIs tend to be much more complex to formulate and manufacture than solution pMDI's.

In accordance with one embodiment of the present invention, a suspension pMDI for apomorphine or its pharmaceutically acceptable salts is provided. Preferably, the propellant of the suspension pMDI is a blend of two commercially available HFA propellants, most preferably about 60% HFA227 (1,1,1,2,3,3,3-heptafluoropropane) and about 40% HFA134a (1,1,1,2-tetrafluoroethane). This approach showed initial physical stability (due to density matching) without addition of further excipients. This is suggestive that such systems are readily manufacturable, although other excipients may be added at low levels to improve pharmaceutical elegance. For example, blends of about 60% HFA227 and about 40% HFA134a were prepared with apomorphine hydrochloride in a 3M coated (Dupont 3200 200) canister with a Bespak BK630 series 0.22 mm actuator. The results of these experiments are discussed below in connection with Example 16.

### Nebulized Systems

Another possible method of administration is via a nebulized system. Such systems include conventional ultrasonic nebulized systems and jet nebulized systems, as well as recently introduced handheld devices such as the Respimat (available from Boehringer Ingelheim) or the AERx (available from Aradigm). In such a system, the apomorphine or a pharmaceutically acceptable salt or ester thereof could be stabilized in a sterile aqueous solution, for example, with antioxidants such as sodium metabisulfite The doses would be similar to those described above, adjusted to take into consideration the lower percentage of apomorphine that will reach the lung in a nebulized system. Although these systems can be used, they are clearly inferior to the DPI systems described above, both in terms of efficiency and convenience of use.

### EXAMPLES

### Example 1

### Preparation of Lactose

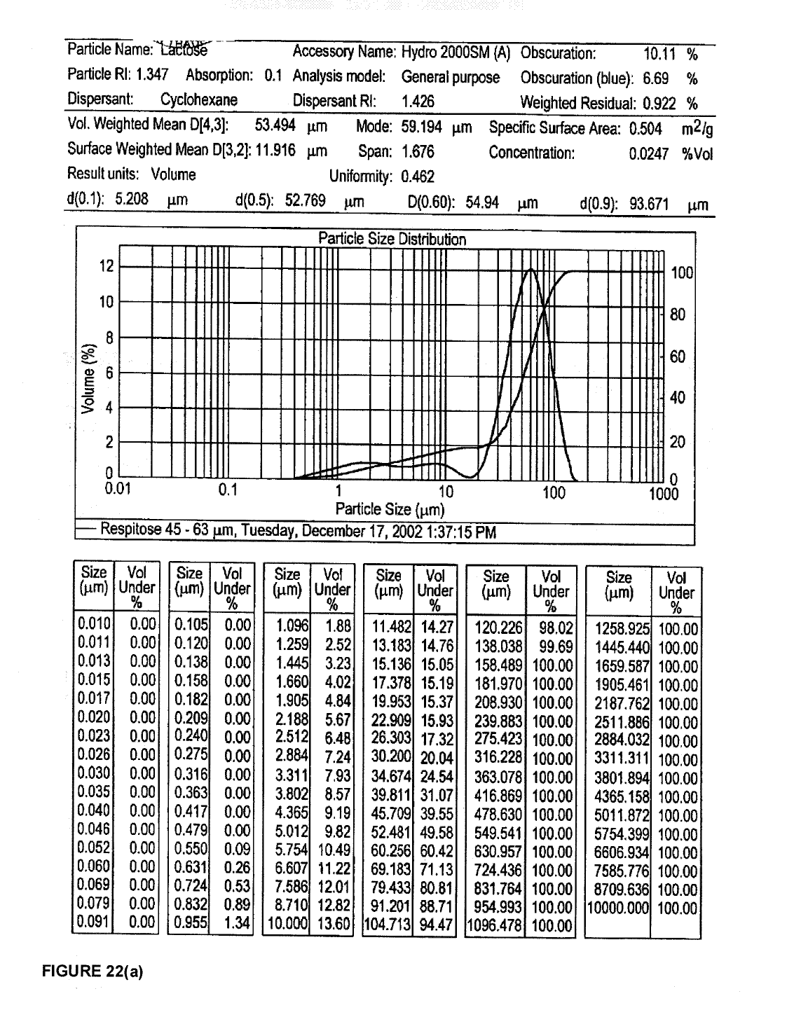

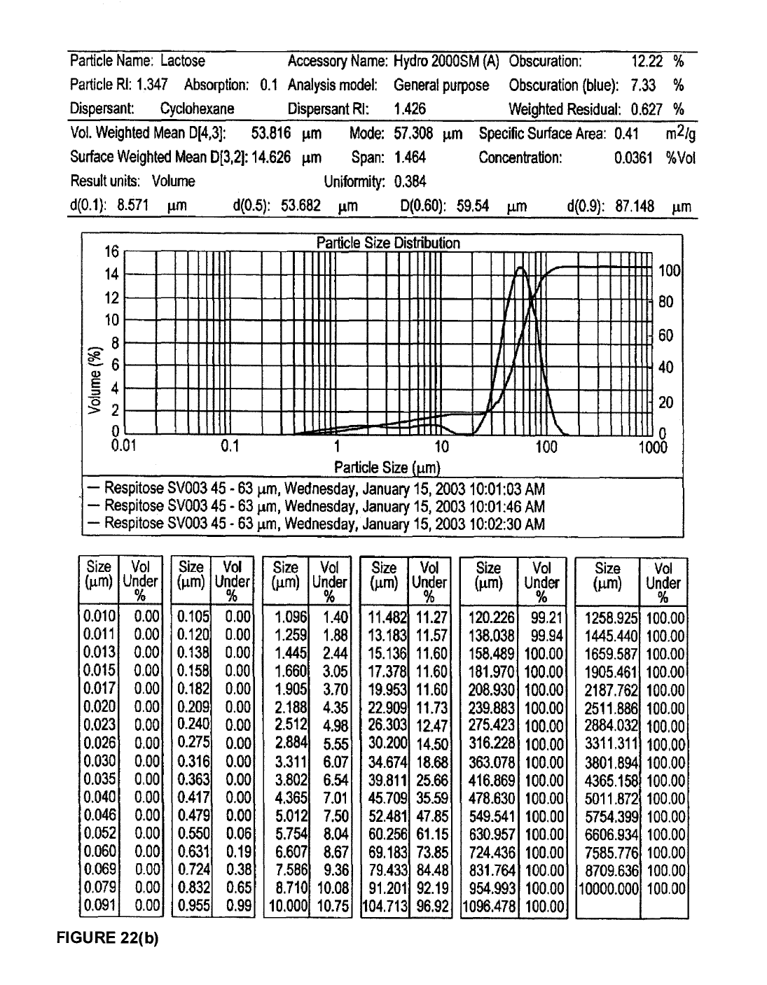

A sieved fraction of Respitose SV003 (DMV International Pharma, The Netherlands) lactose is manufactured by passing bulk material through a 63 μm sieve. This material is then sieved through a 45 μm screen and the retained material is collected. FIGS. 22(A) and 22(B) show the results of a particle size analysis of two batches of the lactose performed with a Mastersizer 2000, manufactured by Malvern Instruments, Ltd. (Malvern, UK). As shown, the lactose had a volume weighted mean of from about 50 to about 55 microns, a d₁₀ of from about 4 to about 10 microns, a d₅₀ of from about 50 to about 55 microns, and a d₉₀ of from about 85 to about 95 microns wherein d₁₀ d₅₀ d₉₀ refer to the diameter of 10%, 50%, and 90% of the analyzed lactose.

### Example 2

### Preparation of Apomorphine-Lactose Formulation

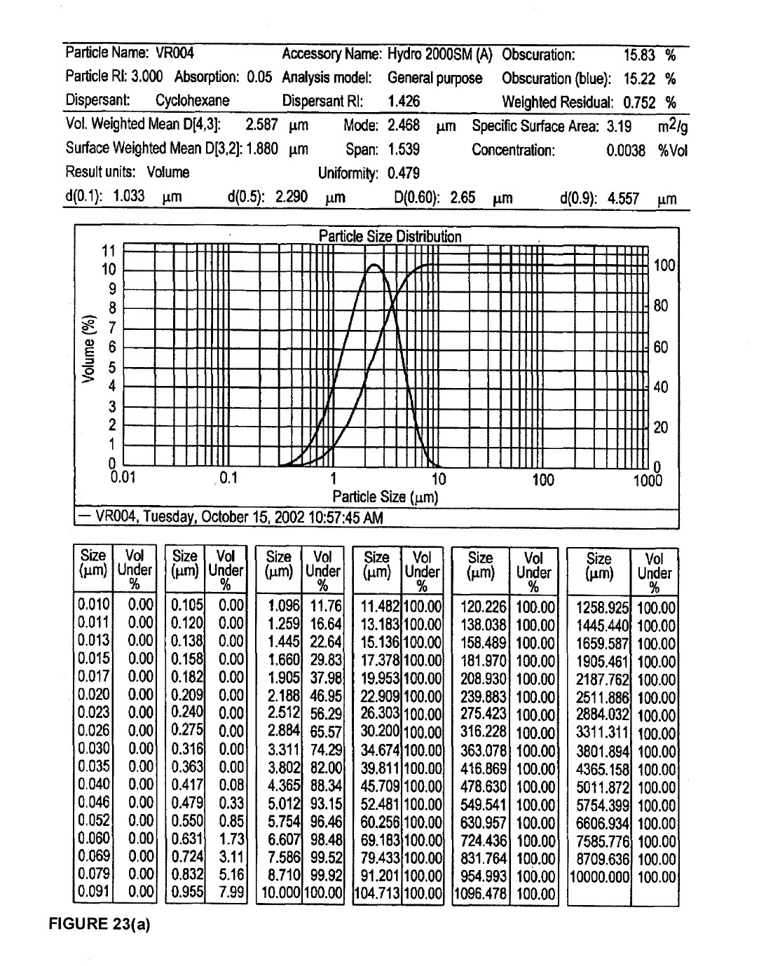

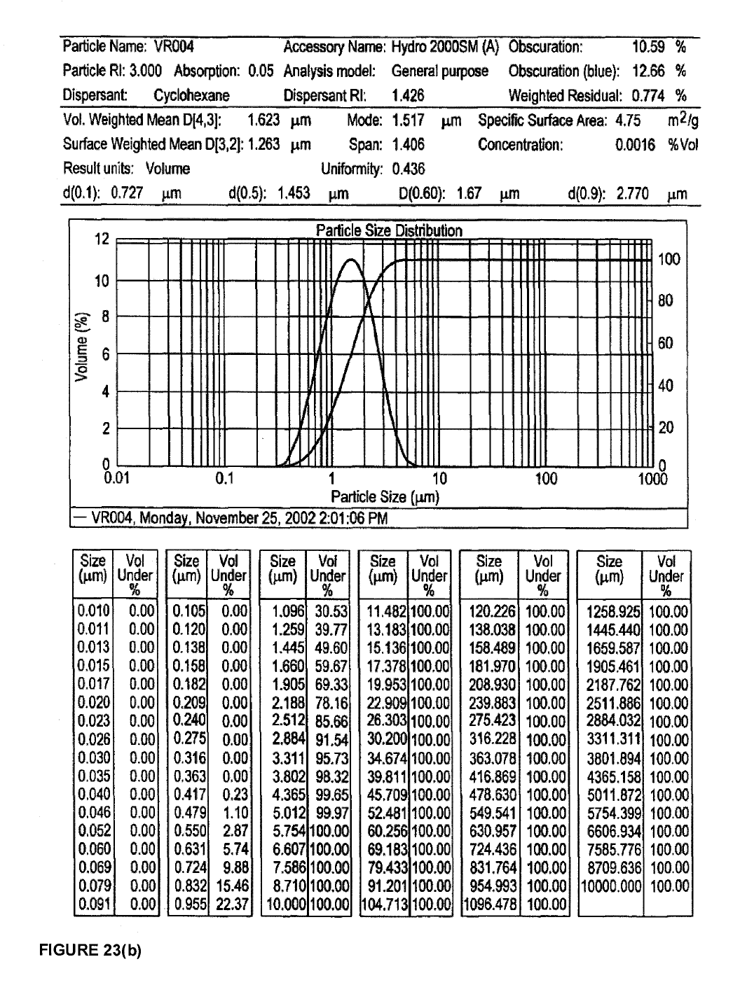

Apomorphine hydrochloride was obtained from Macfarlan Smith Ltd, and was micronized according to the following product specification: >=99.9% by mass<10 microns, based upon a laser diffraction analysis. Actual typical results of the laser fraction analysis were as follows: d₁₀<1 micron, d₅₀: 1-3 microns; d₉₀<6 microns, wherein d₁₀ d₅₀ d₉₀ refer to the diameter of 10%, 50%, and 90% of the analyzed apomorphine hydrochloride. The apomorphine hydrochloride was micronized with nitrogen, (rather than the commonly employed air) to prevent oxidative degradation. FIGS. 23(A) and 23(B) show the results of a particle size analysis of two batches of the micronized apomorphine hydrochloride performed with the Mastersizer 2000, manufactured by Malvern Instruments, Ltd. (Malvern, UK).

### Example 2(a)

### Preparation of 200 Microgram Formulation

70 grams of the lactose of Example 1 was placed into a metal mixing vessel of a suitable mixer. 10 grams of the micronized apomorphine hydrochloride were then added. An additional 70 grams of the lactose of Example 1 was then added to the mixing vessel, and the resultant mixture was tumbled for 15 minutes. The resultant blend was then passed through a 150 μm screen. The screened blend (i.e. the portion of the blend that passed through the screen) was then reblended for 15 minutes.

The particle size distribution of the apomorphine-lactose powder, as determined by an Andersen Cascade Impactor (U.S.P. 26, chapter 601, Apparatus 3 (2003)), showed that the drug particles were well dispersed. In particular, the particle size distribution for a 200 μg dose was as follows:

| | | |
|---|---|---|
| Fine particle dose (<5 μm) | 117 | μg |
| Ultrafine particle dose (<2.5 μm) | 80 | μg |
| MMAD (Mass Median Aerodynamic Diameter) | 1.94 | μm |

### Example 2(b)

### Preparation of 100 Microgram Formulation

72.5 grams of the lactose of Example 1 was placed into a metal mixing vessel of a suitable mixer. 5 grams of the micronized apomorphine hydrochloride were then added. An additional 72.5 grams of the lactose of Example 1 was then added to the mixing vessel, and the resultant mixture was tumbled for 15 minutes. The resultant blend was then passed through a 150 μm screen. The screened blend (i.e. the portion of the blend that passed through the screen) was then reblended for 15 minutes.

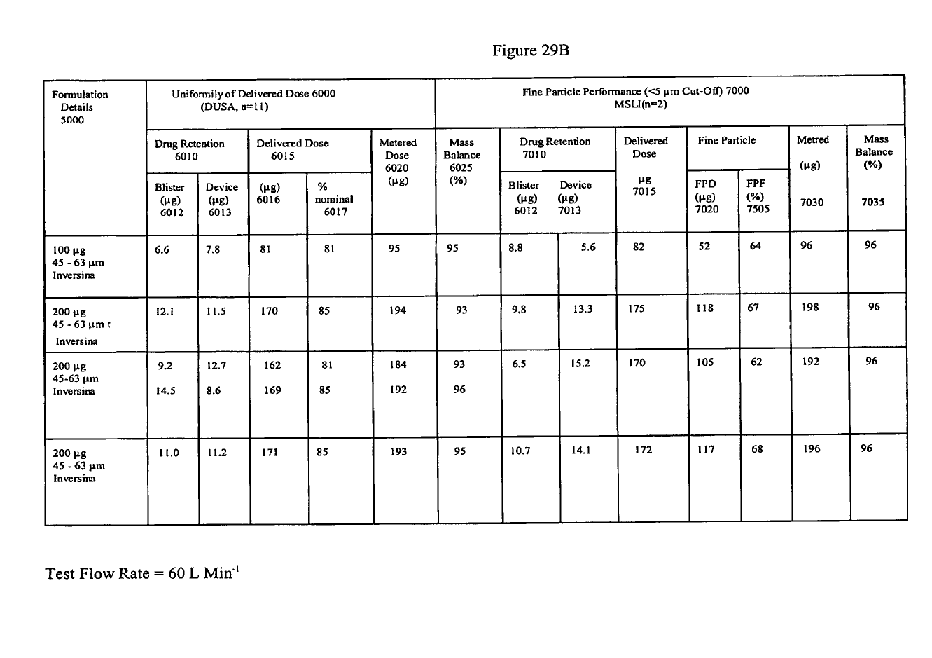

As described below with reference to FIGS. 29(A) and 29(B), in certain batches of Examples 2(a) and 2(b), the mixer used was an Inversina Variable Speed Tumbler Mixer, which is a low shear mixer distributed by Christison Scientific Equipment Ltd of Gateshead, U.K. In other batches, the mixer used was a Retch Grindomix mixer is a higher shear mixer which is also distributed by Christison Scientific Equipment Ltd. Disaggregation was shown to be sensitive to the intensity of the mixing process but a consistent fine particle fraction (about 60%) was obtained using a low shear mixer equipped with a metal vessel such as the Inversina mixer referenced above.

### Example 3

### Incorporation of Formulation into Blisters

The formulations of Example 2(a) and 2(b) were each incorporated into blisters in the following manner. Three milligrams of the apomorphine-lactose formulation were placed in each blister. As described above in connection with FIG. 1, the base of each blister is a cold-formed aluminum blister, formed from a laminate of oriented polyamide (exterior), 45 microns of aluminum (center), and PVC (interior). The lid of the blister is made of a hard-rolled 30 micron lidding foil, having a heat seal laquer. After the formulation is loaded into the interior of the blisters, the blisters are sealed by placing the lid over the blister base, and heat sealing the lid to the base via the heat seal laquer.

### Example 4

### Stability Data

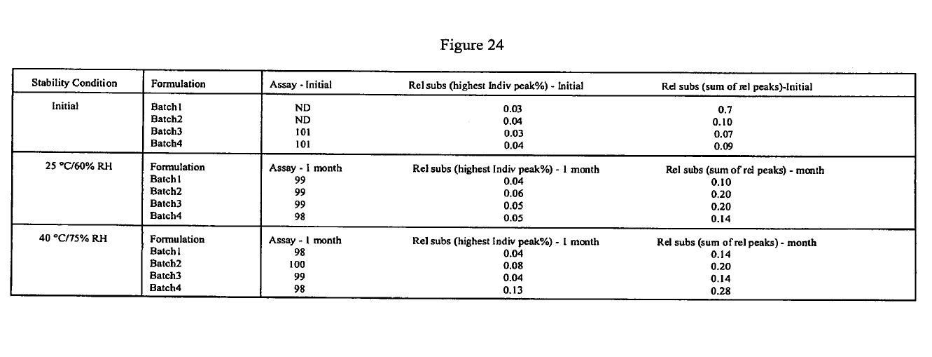

The above-referenced blisters containing the apomorphine-lactose formulations of Example 2(a) were placed into aluminum bags to replicate patient packs, and stored for one month at 25 C and 60% relative humidity, and for one month at 40 C and 75% relative humidity (accelerated storage conditions). The formulation was then removed from the blisters and tested using High Performance Liquid Chromatography (HPLC). The results are shown in FIG. 24. The assay value is the percent of the expected apomorphine content of the formulation, the “Rel Subs (highest individual peak %)” is the largest related substance peak as a percentage of the total peaks in the chromatogram; and the “Rel Subs (sum of related substance peaks)” is the total related substance peaks as a percentage of the total peak area in the chromatogram. As one of ordinary skill in the art will appreciate, these values are well within the acceptable parameters of 0.2% for Rel Subs (highest individual peak %) and 1.0% for Rel Subs (sum of related substance peaks).

### Example 5

### Inhalation Testing

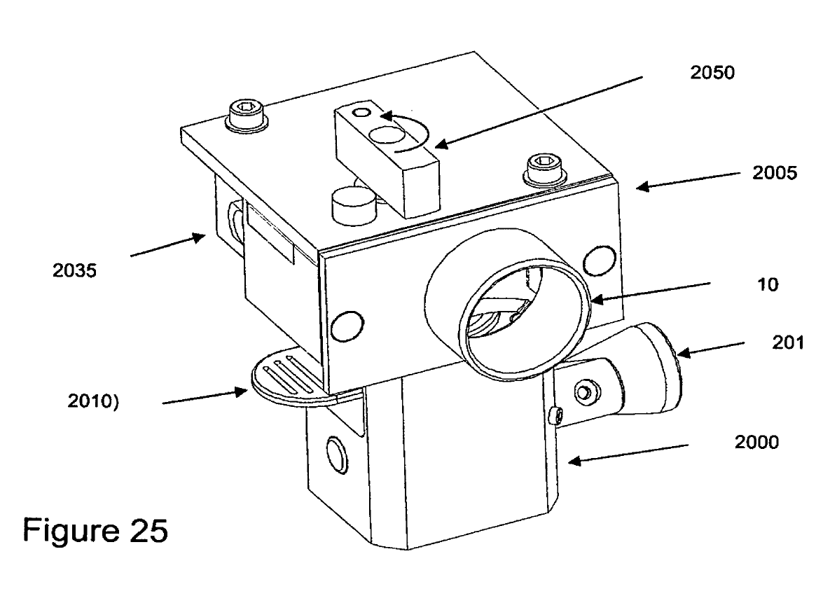

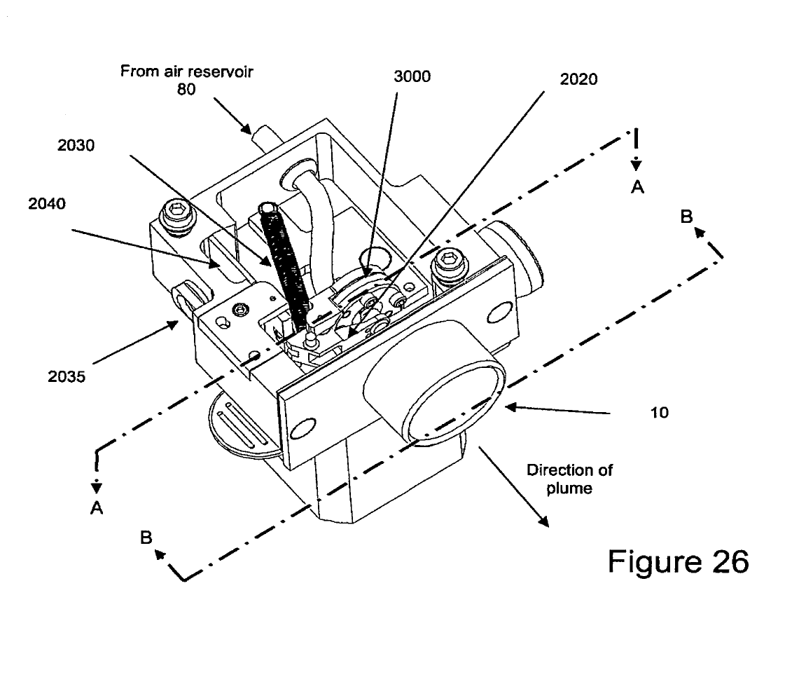

The above referenced blisters containing the 100 and 200 microgram apomorphine-lactose formulations were subjected to testing using the prototype inhaler shown in FIGS. 25 through 28. Referring to FIGS. 25 and 26, the inhaler comprises a reservoir 80 (not shown) which provides a charge of compressed air, a base block 2000, an airway 2004, a mouthpiece 10 through which the dose is inhaled, a blister loader 2010 by which the dose is presented to the inhaler, a crank arm 2015 by which the dose blister (60-70) is pierced, a vortex nozzle 3000 for aerosolizing the dose, and an exit valve 2020 by which the aerosolized dose is released into the mouthpiece 10.

In use, the user places a foil blister (not shown) onto the blister loader 2010 and inserts the blister loader into the device in the position shown in FIG. 25. The user then pierces the blister by moving the crank arm 2015 from a rest position to a pierce position in which it locks. The reservoir 80 is then charged from a compressed air line (not shown) such that the reservoir 80 contains a volume of pressurized air (typically 15 ml) at a relatively low pressure (typically 1.5 bar gauge). The compressed air is prevented from leaving the device by the valve 2020 at the exit to the vortex nozzle 3000. The device is now primed to deliver the dose.

When the user inhales via the mouthpiece, breath actuation vane 2025 moves, opening the exit valve 2020 and releasing the compressed air in the reservoir. The air flows through the blister, entraining the dose of powder and carrying it to the vortex nozzle 3000. In the nozzle the powder experiences high centrifugal and shear forces which deagglomerate the dose before delivering it to the user via the mouthpiece 10 as a finely dispersed aerosol.

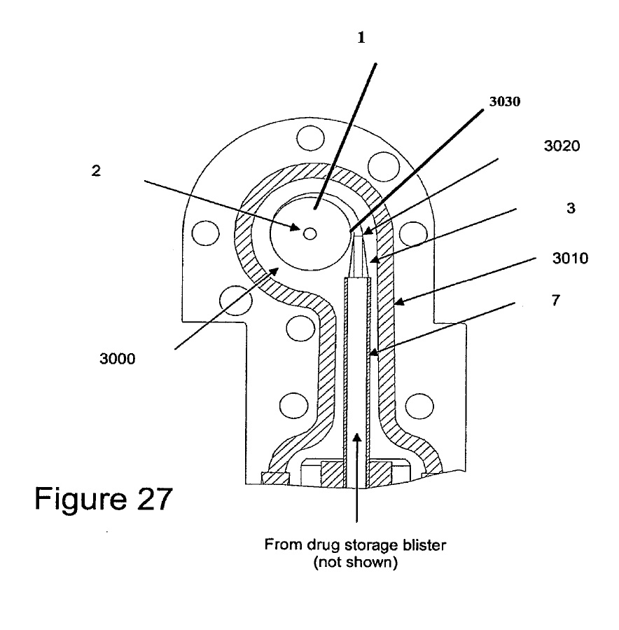

Referring to FIG. 27, the vortex nozzle 3000 comprises an inlet conduit 3, a vortex chamber 1, an outlet port 2 and a nozzle seal 3010. In use, the compressed gas and entrained dry powder dose from the blister (not shown) enters the vortex chamber via an inlet tube 7 and inlet conduit 3 and leaves the nozzle 3000 via the exit port 2. At the point 3020 where the inlet conduit 3 joins the vortex chamber 1, the outer wall of the chamber has a radius of 3.35 mm. Continuing counter-clockwise along the wall of the chamber 1 for 180 degrees, the radius of the chamber reduces linearly to 2.5 mm at point 3025. The radius is then constant at 2.5 mm as the wall of the chamber continues in counter-clockwise direction until it intersects the inlet conduit. The height of the vortex chamber is 1.6 mm. The inlet tube 7 has an internal diameter of 1.22 mm and feeds into the inlet conduit 3.

The inlet conduit 3 tapers in section from a 1.22 mm diameter where it joins the inlet tube 7 to its narrowest point where the inlet conduit 3 joins the vortex chamber 1 and has a height of 1.1 mm high and a width of 0.5 mm. As such, the inlet conduit 3 does not extend to the full height of the vortex chamber, which is 1.6 mm. The outlet port 2 diameter is 0.7 mm and the thickness of the outlet port 2 is 0.35 mm.

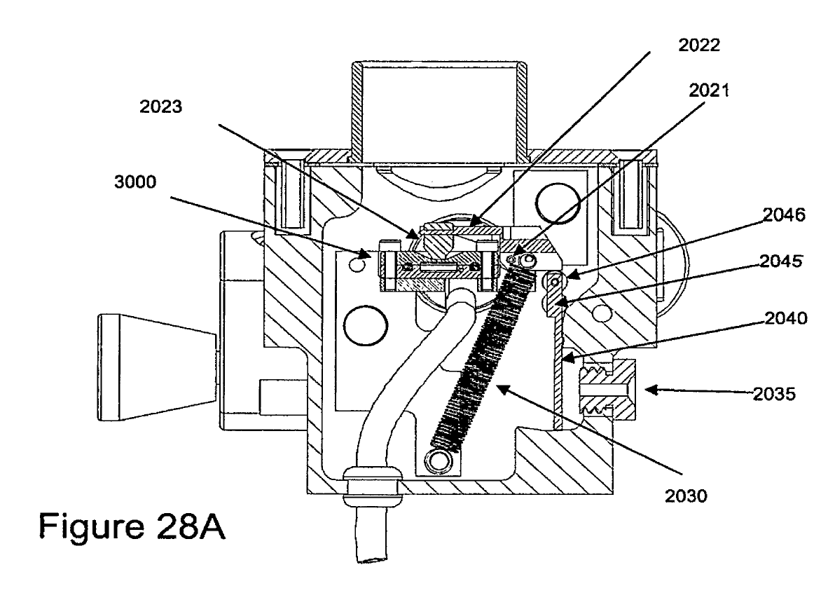

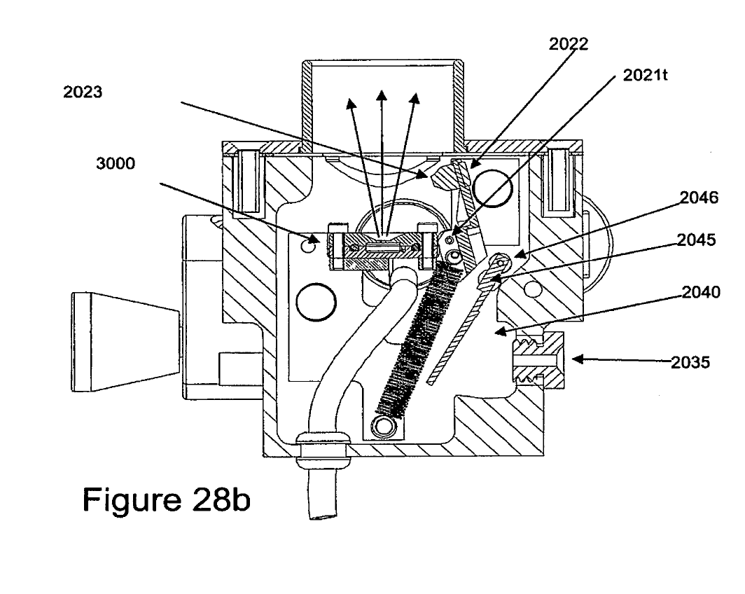

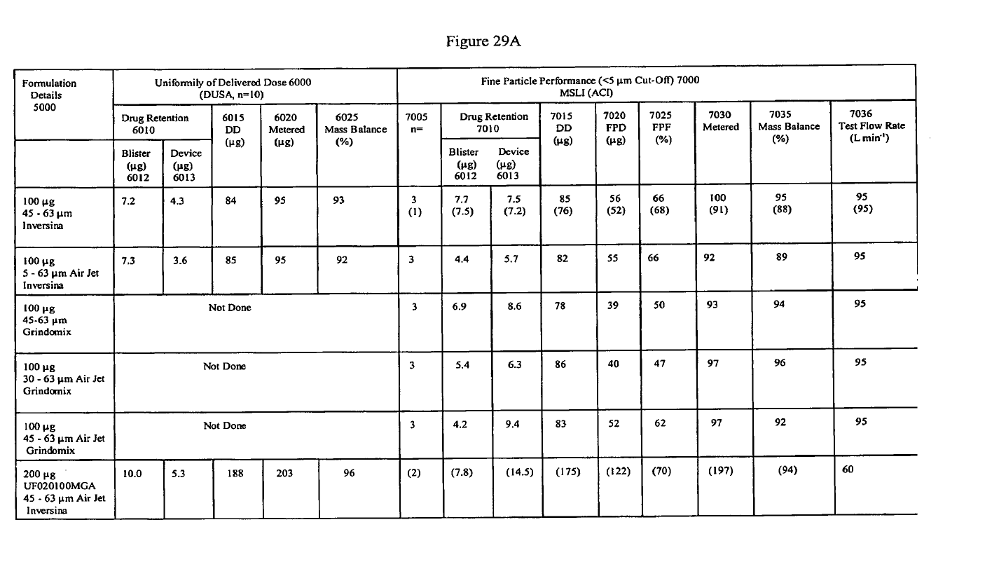

Referring to FIGS. 26, 28A and 28B, the breath actuated mechanism comprises a valve 2020 at the outlet port of the vortex nozzle, a valve spring 2030 biased to open the valve, a breath actuation vane 2040 that rotates in response to inhalation by a user, and an inspiratory air inlet 2035 through which air is drawn when a user inhales through the mouthpiece 10. The valve 2020 includes a resilient valve seal 2023 mounted on a valve arm 2022 which in turn is rotatably mounted on a valve arm pivot 2021. When the valve arm 2022 is in the closed position (FIG. 28A), the valve seal 2023 covers and seals the vortex nozzle outlet port 2. In the open position (FIG. 28B) the vortex nozzle outlet port 2 is open to allow the dose to exit the nozzle 3000.

The breath actuation vane 2040 is rotatably mounted on a vane pivot 2045. The vane 2040 includes a vane roller 2046 which is rotatably mounted on the vane 2045 and is free to rotate, and a vane return spring (not shown) which biases the vane 2040 to the closed position as shown in FIG. 28A. When the valve 2040 is in the closed position, the valve seal 2023 is compressed to seal the nozzle outlet 2 and the opposite end of the valve arm 2022 rests on the vane roller 2046 and is prevented from rotating.

When a user inhales through the mouthpiece 10, air flows into the airway via the inspiratory air inlet 2035. This flow and the pressure drop it generates across the breath actuation vane 2040 cause the vane 2040 to rotate about its pivot 2045. The vane roller 2046 rolls against the end of the valve arm 2022 and then becomes clear of the valve arm 2022 as the vane 2040 rotates further. This allows the valve arm 2022 to rotate under the influence of the valve spring 2030, which removes the valve seal 2023 from the output port 2 (i.e., opening the valve) to release the dose from the nozzle as shown in FIG. 28B.

The breath actuated mechanism can be reset for the next dose by rotating the valve reset lever 2050 through 90 degrees and then returning it to its original position. The reset lever 2050 acts on the valve arm 2022 to close the valve (by causing the valve seal 2023 to cover output port 2) and allow the breath actuation vane 2040 to return to its closed position under the influence of the vane return spring (not shown).

In order to obtain the inhalation data described below, the inhaler of FIGS. 25 through 28 was used in conjunction with three instruments, a Multi-Stage Liquid Impinger (MSLI) (U.S.P. 26, chapter 601, Apparatus 4 (2003), an Anderson Cascade Impactor (ACI) (U.S.P. 26, chapter 601, Apparatus 3 (2003), and a Dosage Unit Sampling Apparatus (DUSA) (U.S.P. 26 chapter 601, Apparatus B (2003). Each of these devices have an input for receiving the mouthpiece 10 of the inhaler of FIGS. 25-28.

The DUSA is used to measure the total amount of drug which leaves the inhaler. With data from this device, the metered and delivered dose is obtained. The delivered dose is defined as the amount of drug that leaves the inhaler. This includes the amount of drug in the throat of the DUSA device, in the measuring section of the DUSA device and the subsequent filters of the DUSA device. It does not include drug left in the blister or other areas of the inhaler of FIGS. 25-28, and does not account for drug “lost” in the measuring process of the DUSA device. The metered does includes all of the drug which leaves the blister.

The MSLI is a device for estimating deep lung delivery of a dry powder formulation. The MSLI includes a five stage cascade impactor which can be used for determining the particle size (aerodynamic size distribution) of Dry Powder Inhalers (DPIs) in accordance with USP 26, Chapter 601 Apparatus 4 (2003) and in accordance with the European Pharmacopoeia., Method 5.2.9.18, Apparatus C, Supplement 2000.

The ACI is another device for estimating deep lung delivery of a dry powder formulation. The ACI is multi-stage cascade impactor which can be used for determining the particle size (aerodynamic size distribution) of Dry Powder Inhalers (DPI) in accordance with USP 26, Chapter 601 Apparatus 3 (2003).

As described below, the MSLI and the ACI testing devices can be used to determine, inter alia, the fine particle dose, or FPD (the amount of drug, e.g., in micrograms, that is measured in the sections of the testing device which correlates with deep lung delivery) and the fine particle fraction, or FPF, (the percentage of the metered dose which is measured in the sections of the testing device which correlates with deep lung delivery).

FIGS. 29(A) and 29(B) illustrate the results of tests performed on the apomorphine-lactose formulation of Example 2, using the inhaler of FIGS. 25-28. The FPD, FPF and MMAD values were generated from the MSLI and ACI data using the Copley Inhaler Data Analysis Software (CITDAS) V1.12. In FIG. 29(a), data is shown for six formulations, which are identified in column 5000. FIG. 29(b) provides data for an additional four formulations. In each Figure, the test data for the formulations is divided into two types: data related to uniformity of the delivered dose for the formulations (column 6000) and data related to fine particle size performance of the formulations (column 7000).

Referring to FIG. 29(a), the first five formulations listed in column 5000 include 3 mg. of the 100 microgram formulation of Example 2(B). The sixth formulation listed includes 3 mg. of the 200 microgram formulation of Example 2(A). The first, second, and sixth formulation listings in 5000 contain the notation “Inversina” to indicate that the mixer used in Example 2 was the Inversina Mixer, and the third, fourth, and fifth formulation listing contain the notation “Grindomix” to indicate that the mixer used in Example 2 was the Grindomix Mixer. The second and fourth formulations listed also contain the notation “Air Jet” to indicate that for these formulations the lactose in Example 1 was sieved with an Air Jet Sieve which applies a vacuum to the screen Sieve apparatus, rather than a conventional screen Sieve (which was used for the first third, fifth, and sixth formulations listed). The fifth formulation listed also contains the notation “20-30 μm Extra Fine” to indicate that the lactose for this formulation was screen sieved through 20 micron and 30 micron screens.

In section 6000 of FIG. 29(a) the DUSA apparatus described above is used to provide data for the formulations regarding the drug retention in the blister (6012), the drug retention in the inhaler (6013), the delivered dose (6015), the metered dose (6020), and the mass balance percentage (6025). The notation n=10 indicates that the inhaler and DUSA apparatus was fired 10 times for each of the three formulations for which DUSA data is listed. The data listed in section 6000 is an average of the 10 firings.

In section 7000 of FIG. 29(a), the fine particle performance is measured with two different devices, the MSLI and the AO. Data for the ACI, where available, is indicated in parenthesis ( ). In any event, the data provided in section 7000 is for particles having a particle size diameter of less than 5 microns (referred to in this discussion as “fine particles”). As such, column 7012 provides the fine particle drug retention in the blister, column 7013 provides the fine particle drug retention in the inhaler, column 7015 provides the amount of fine particles in the delivered dose, column 7020 provides the FPD for the formulation, column 7025 provides the FPF for the formulation, column 7015 provides the amount of fine particles in the metered dose, column 7035 provides the mass balance percentage for the formulations in the MSLI (ACI) tests, and column 7036 provides the test flow rate for the formulations. Column 7005 indicates that the number of times the inhaler and MSLI (or ACI) apparatus were fired, and the data listed is an average of the “n” firings.

FIG. 29(b) is similar to FIG. 29(a), with similar items bearing identical reference numbers. The first formulation listed in column 5000 include 3 mg. of the 100 microgram formulation of Example 2(b), the remaining four formulations include 3 mg. of the 200 microgram formulation of Example 2(a), and all of the formulations were made with the Inversina Mixer, and were sieved with 43 and 63 micron screens. The DUSA data in column 6000 was obtained in the same manner as in FIG. 29(a), except that n=11. All of the fine particle performance data in section 7000 was obtained using the ACI apparatus with n=2, and a flow rate of 60 L min⁻¹.

As illustrated in FIGS. 29(a) and 29(b), when the formulations were mixed using the low shear Inversina mixer, the fine particle fraction (FPF) ranged from a low of 62% to a high of 70%, and the percent delivered dose ranged from a low of 81% to a high of 94%. The formulations made with the higher shear Grindomix mixer exhibited a fine particle fraction of from 47% to 50% for formulations including the 43-63 micron lactose. The formulation made with the high shear Grindomix mixer and with lactose sieved at 20 and 30 microns exhibited an increased fine particle fraction of 62%.

### Example 6

### Preparation of 400 Microgram Formulation in 3 Mg Blister (Prophetic)

A 400 microgram formulation can be manufactured in the manner set forth above with regard to Example 2, with the components provided in the following amounts:

| Composition | Amount (μg) | Percent |
|---|---|---|
| Apomorphine Hydrochloride | 400 | 13.33 |
| Lactose | 2600 | 86.66 |
| Total | 3000 | 100 |

### Example 7

### Preparation of 600 Microgram Formulation in 3 Mg Blister

A 600 microgram formulation can be manufactured in the manner set forth above with regard to Example 2, with the components provided in the following amounts:

| Composition | Amount (μg) | Percent |
|---|---|---|
| Apomorphine Hydrochloride | 600 | 20 |
| Lactose | 2400 | 80 |
| Total | 3000 | 100 |

Although the above referenced examples utilize a blister “fill weight” of 3 mg, it should be appreciated that larger or, smaller fill weights may also be used. For example, in Examples 8-12 below, fill weights of 1 mg or 2 mg are provided. Although a variety of techniques for filling blisters to such fill weights may be used, it is believed that commercial production of blisters with 1 mg and 2 mg fill weights can be achieved with a Harro-Hoefliger Omnidose Drum Filler. Lower fill weights, and in particular fill weights on the order of 1 mg, are believed to provide superior fine particle fractions as compared to higher fill weights. For example, in experiments performed using an ACI with a single “shot”, a 200 microgram apomorphine hydrochloride formulation as described above provided a fine particle fraction of 73% with a 3 mg fill weight, 71% with a 2 mg fill weight, and 83% with a 1 mg fill weight.

### Example 8

### Preparation of 800 Microgram Formulation in 2 Mg Blister (Prophetic)

An 800 microgram formulation can be manufactured in the manner set forth above with regard to Example 2, with the components provided in the following amounts:

| Composition | Amount (μg) | Percent |
|---|---|---|
| Apomorphine Hydrochloride | 800 | 26.66 |
| Lactose | 1200 | 73.33 |
| Total | 2000 | 100 |

### Example 9

### Preparation of 200 Microgram Formulation with Magnesium Stearate in 1 Mg Blister (Prophetic)

A 200 microgram formulation with Magnesium Stearate with the components provided in the following amounts:

| Composition | Amount (μg) | Percent |
|---|---|---|
| Apomorphine Hydrochloride | 200 | 20.00 |
| Lactose | 797.5 | 79.75 |
| MgStearate | 2.5 | 00.25 |
| Total | 1000 | 100 |

This formulation is prepared in the manner set forth above with regard to Example 2, except that Magnesium Stearate is added to the mixture along with the apomorphine hydrochloride.

### Example 10

### Preparation of 400 Microgram Formulation With Leucine in 2 Mg Blister (Prophetic)

A 400 microgram formulation with Leucine with the components provided in the following amounts:

| Composition | Amount (μg) | Percent |
|---|---|---|
| Apomorphine Hydrochloride | 400 | 20 |
| Lactose | 1560 | 78 |
| Micronized Leucine | 40 | 2 |
| Total | 2000 | 100 |

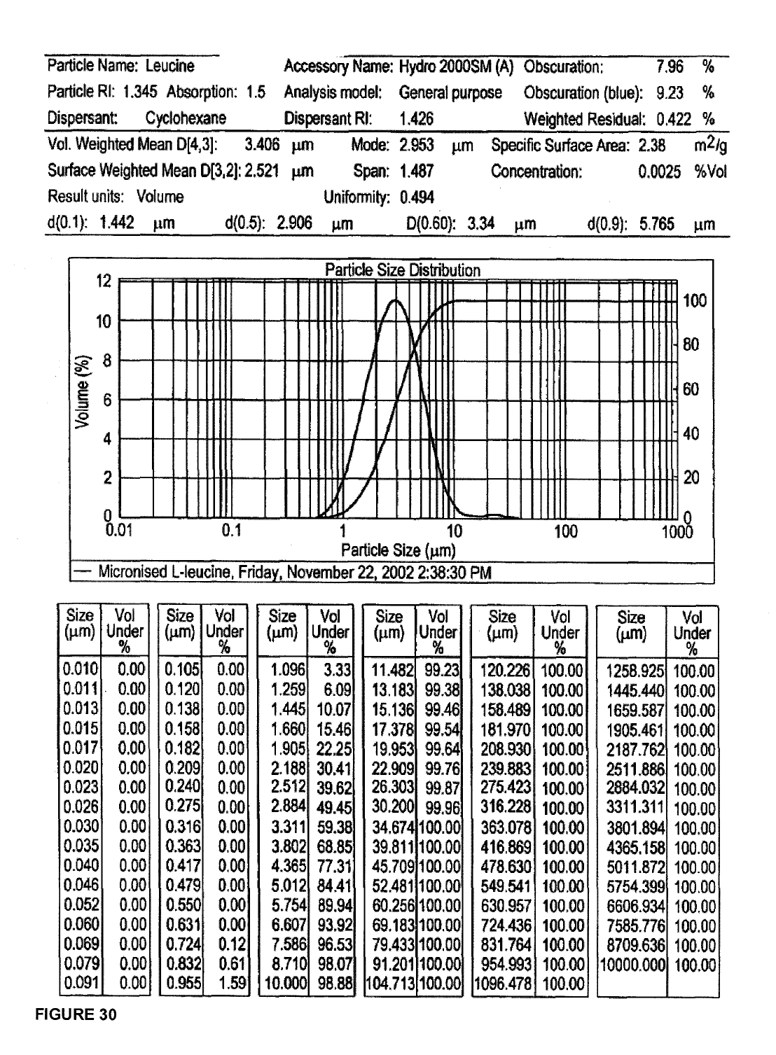

This formulation is prepared in the manner set forth above with regard to Example 2, except that micronized leucine is added to the mixture along with the apomorphine hydrochloride. FIG. 30 shows the results of a particle size analysis of a preferred micronized Leucine performed with the Mastersizer 2000, manufactured by Malvern Instruments, Ltd. (Malvern, UK). As illustrated, the exemplified micronized leucine has a volume weighted mean particle diameter of 3.4 microns, with 90% of the particles having a volume weighted mean particle diameter of less than 6 microns.

### Example 11

### Preparation of 200 Microgram Formulation in 2 Mg Blister (Prophetic)

A 200 microgram formulation can be manufactured in the manner set forth above with regard to Example 2, with the components provided in the following amounts:

| Composition | Amount (μg) | Percent |
|---|---|---|
| Apomorphine Hydrochloride | 200 | 10 |
| Lactose | 1800 | 90 |
| Total | 2000 | 100 |

### Example 12

### Preparation of 200 Microgram Formulation in 1 Mg Blister (Prophetic)

A 200 microgram formulation can be manufactured in the manner set forth above with regard to Example 2, with the components provided in the following amounts:

| Composition | Amount (μg) | Percent |
|---|---|---|
| Apomorphine Hydrochloride | 200 | 20 |
| Lactose | 800 | 80 |
| Total | 1000 | 100 |

### Example 13

### Preparation of 400 Microgram Formulation in 2 Mg Blister (Prophetic)

A 400 microgram formulation can be manufactured in the manner set forth above with regard to Example 2, with the components provided in the following amounts:

| Composition | Amount (μg) | Percent |
|---|---|---|
| Apomorphine Hydrochloride | 400 | 20 |
| Lactose | 1600 | 80 |
| Total | 2000 | 100 |

### Example 14

### In Vivo Clinical Data-Patients Treated with Apomorphine Via DPI Inhalation

In this study, 35 patients were treated with 4 random doses of either placebo, 200 μg of apomorphine hydrochloride, 400 μg of apomorphine hydrochloride, or 800 μg of apomorphine hydrochloride. The doses were administered either with the blister of Example 3 (200 micrograms of apomorphine hydrochloride in a 3 mg blister) or in a placebo blister (200 micrograms of placebo in the 3 mg blister of Example 3). During each treatment, a patient took the given dose and was left alone to watch an hour of visual sexual stimulation (VSS). At 50-55 minutes after administration, the patients were warned that the study would end at 60 minutes. After 60 minutes, the patient's were asked to rate the quality and duration of their response to VSS. In this regard, the quality of response is defined as one of four grades: 0: no effect; 1: some tumescence, no rigidity; 2: some tumescence, some rigidity, but not suitable for penetration; 3: rigidity and tumescence that would enable penetration but is not complete erection; 4: complete erection. This study was conducted in a double blind fashion, where both the healthcare professional administering the treatment and the patient were not informed as to the actual dose being administered. The patients who participated in this study were randomized. During each treatment, each of the 35 patients received 4 blisters regardless of the dose i.e., a patient receiving a 400 μg HCl dose would receive 2 (two) of the apomorphine HCl blisters and 2 (two) of the placebo blisters and a patient receiving only placebo took 4 (four) of the placebo blisters. The study showed that the groups treated with 400 μg and 800 μg of apomorphine HCl experienced the quickest onset of effect, longest duration and most complete erections as compared to the groups treated with either Placebo or 200 μg apomorphine HCl dose. For example, the group treated with 800 μg apomorphine HCl exhibited a median onset of effect in about 8 or less minutes after administration of apomorphine HCl as compared to about 11 or less minutes for the 200 μg apomorphine HCl group, based upon grade 3 and 4 responders. Grade 3 or 4 responses were achieved as quickly as 4 minutes for the 400 and 800 μg groups. It is believed that if this treatment were to be repeated with single dosing as opposed to 4 doses at a time (i.e. one 800 μg blister dose), the response to treatment would exhibit an even faster onset, thereby, providing even more effective treatment.

In the study, patients treated with placebo (4 blisters, each consisting of placebo) showed a 31.4% average response rate. The 200 μg group (4 blisters, 1 containing 200 μg apomorphine HCl and the remaining 3 blisters each containing placebo) showed a 22.9% average response rate, the 400 μg group (4 blisters, 2 containing 200 μg apomorphine HCl and the remaining 2 containing placebo) showed a 48.5% average response rate, and the 800 μg group (4 blisters, each containing 200 μg apomorphine HCl) showed a 58.8% average response rate. As the patients treated with 400 μg and 800 μg displayed significantly higher response rates as compared to those patients treated with either placebo or 200 μg, the 400 μg and 800 μg doses are considered to be effective. (See table 4 below).

TABLE 4
SUMMARY OF RESPONSE RATE (ITT POPULATION)

| Dose | Evaluated | Responding | Rate (%) | CI Limit¹ | Effective? |
|---|---|---|---|---|---|
| Placebo | 35 | 11 | 31.4% | 18.7% | No |
| 200 μg | 35 | 8 | 22.9% | 11.9% | No |
| 400 μg | 33 | 16 | 48.5% | 33.3% | Yes |
| 800 μg | 34 | 20 | 58.8% | 43.3% | Yes |

¹The confidence interval (CI) is a one sided 95% CI. It extends from the limit shown to 100%.

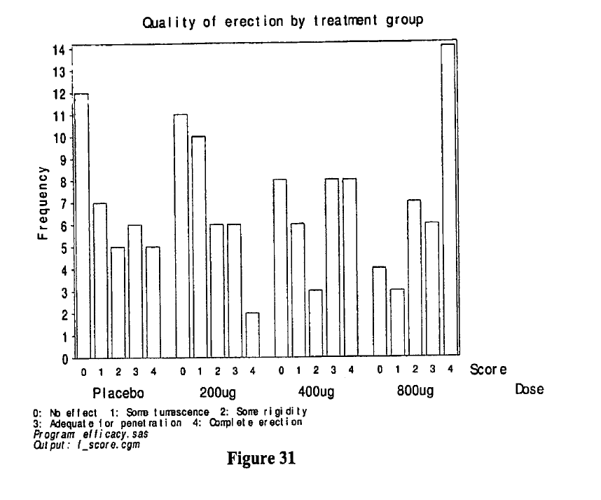

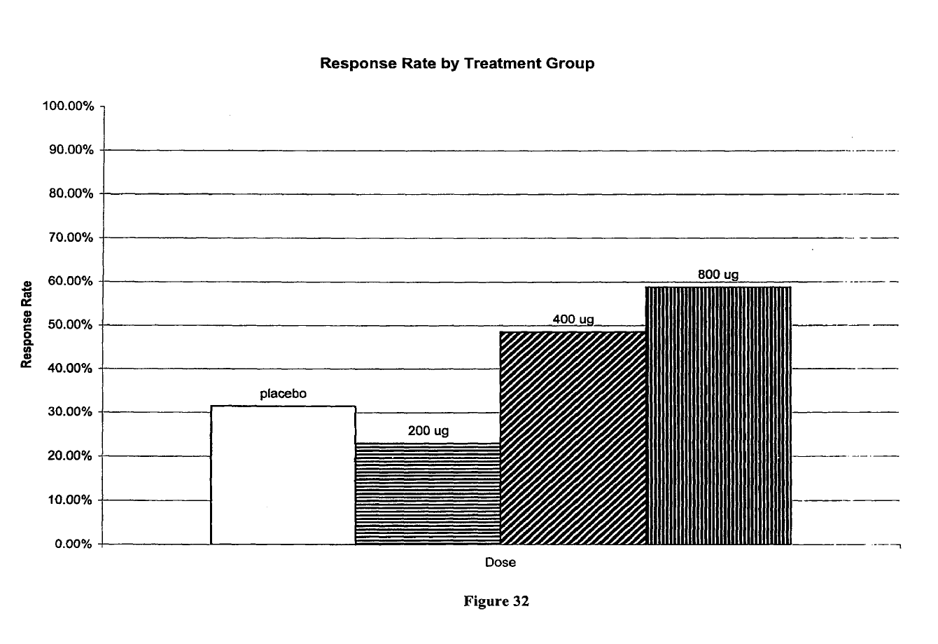

The primary measure of efficacy, as defined in the protocol, was the proportion of subjects reporting a grade 3 or 4 erection, using the criteria defined in the International Index of Erectile Function (IIEF). Grade 3 and 4 erections are regarded as “sufficient for successful intercourse”. Using these criteria, the 400 μg and 800 μg doses of apomorphine HCL were deemed effective. As illustrated in FIGS. 31 and 32, a clear dose response relationship was noted amongst the active dose groups, both in the proportion of “sufficient” erections, the proportion of grade 4 or “full” erections and response rate. For example, the group treated with 800 μg of apomorphine HCl showed the greatest number of grade 4 erections, highest response rate, quickest onset of effect and longest duration in comparison to the groups treated with Placebo, 200 μg and 400 μg of apomorphine HCl.

With respect to efficacy, table 5 below illustrates that the 200 μg apomorphine HCl dose group exhibited a median onset of effect of 11 minutes after administration (with a standard of deviation of 4.2), and the placebo group exhibited a median onset of effect of 10 minutes after administration (with a standard of deviation of 7.8). In contrast, the 400 μg and 800 μg apomorphine HCl dose groups exhibited the quickest median onset of effect (8 (SD 7.5) and 8 (SD 5.0) respectively). The 400 μg and 800 μg apomorphine HCl dose groups also exhibited the most complete erections, longest duration and highest response rate percentages as compared to the groups treated with either 200 μg apomorphine HCl or placebo.

TABLE 5
Summary of efficacy
(ITT population)

| | | Treatment | | | |
|---|---|---|---|---|---|
| | Quality | | 200 μg | 400 μg | 800 μg |
| Quality | Grade | Placebo | Apo. | Apo | Apo. |
| No effect | 0 | 12 | 11 | 8 | 4 |
| Some tumescence, | 1 | 7 | 10 | 6 | 3 |
| no rigidity | | | | | |
| Some tumescence and | 2 | 5 | 6 | 3 | 7 |
| rigidity | | | | | |
| Partial erection | 3 | 6 | 6 | 8 | 6 |
| Full erection | 4 | 5 | 2 | 8 | 14 |
| Onset (min post dose) | N | 11 | 8 | 16 | 19 |
| | Mean | 13 | 13 | 11 | 10 |
| | SD | 7.8 | 4.2 | 7.5 | 5.0 |
| | Min | 4 | 8 | 3 | 3 |
| | Max | 27 | 20 | 28 | 17 |
| | Median | 10 | 11 | 8 | 8 |
| Duration (min) | N | 11 | 8 | 16 | 19 |
| | Mean | 29 | 33.3 | 31.1 | 31.2 |
| | SD | 18.0 | 7.7 | 18.4 | 16.6 |
| | Min | 6 | 24 | 4 | 6 |
| | Max | 52 | 47 | 54 | 54 |
| | median | 30.0 | 31.5 | 38 | 36 |

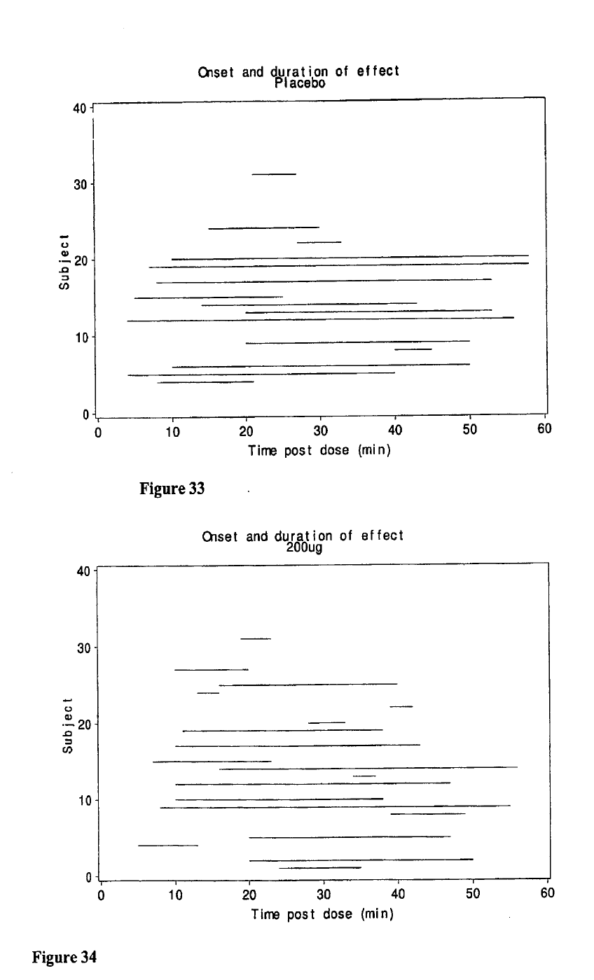

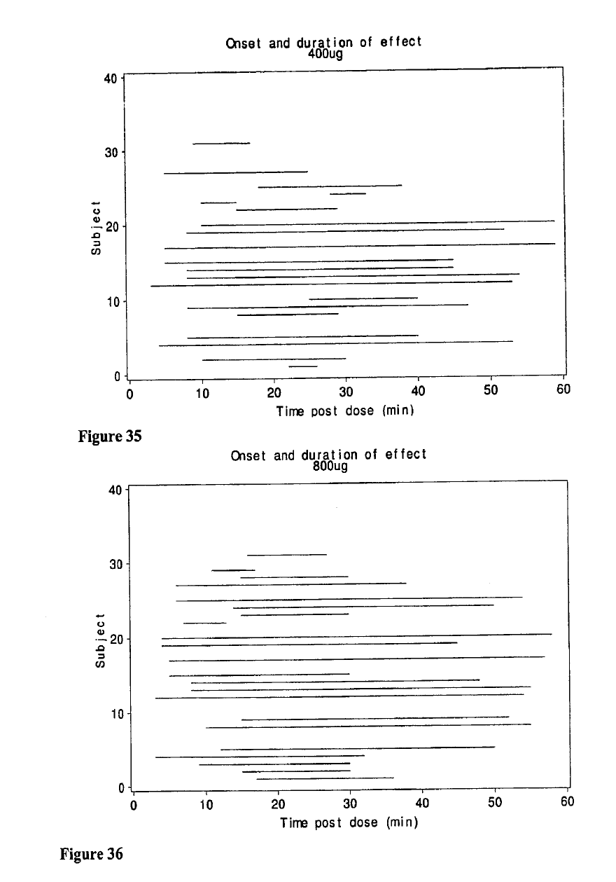

A more detailed illustration of the onset and duration of effect for each individual group is provided in FIGS. 33 through 36. FIG. 33 shows the onset and duration of effect for the patients who were treated with placebo. FIG. 34 shows the onset and duration of effect for the patients treated with 200 μg apomorphine HCl. FIG. 35 shows the onset and duration of effect for the patients treated with 400 μg apomorphine HCl and FIG. 36 shows the onset and duration of effect for the patients treated with 800 μg apomorphine HCl. For example, referring to FIG. 36, it is apparent that one patient in the 800 μg apomorphine HCl group experienced the onset of an erection at about 4 minutes after administration and lasted for about 54 minutes. Referring to FIG. 35, for example, it is apparent that a patient in the 400 μg apomorphine HCl group experienced the onset of an erection at about 3 minutes after administration and lasted for about 50 minutes. In contrast, FIG. 34 shows that one patient in the 200 μg group experienced the onset of an erection at about 40 minutes after administration and lasted for about 3 minutes. Overall, these figures illustrate that the groups that received 400 μg and 800 μg doses of apomorphine HCl experienced faster onset of erections and longer duration. It should be appreciated that the testing period lasted 60 minutes, and the patients were reminded at 50-55 minutes that the test would end at 60 minutes. As such, it is possible that the duration of erection would, in some cases, have extended past 54 minutes, absent the impending termination of the test at 60 minutes.

Adverse events were monitored during each dosing period. The proportion of patients experiencing one or more adverse events was similar in all four treatment groups. No serious adverse events were observed and no adverse event led to the premature discontinuation of any subject. All adverse events were mild or moderate in severity and occurred in a small percentage of the groups treated. Table 6 is a summary of all adverse events. Table 7 is a summary of all treatment related adverse events, and Table 8 breaks treatment related adverse events down by body system. Referring to Table 6, only 6% of the 800 μg apomorphine HCl group experienced adverse events, which is the same percentage of those who experienced adverse events in both the placebo and 200 μg apomorphine HCl group. Referring to Table 8, adverse events were most frequently observed in the Respiratory, thoracic and mediastinal disorders body systems.

TABLE 6
Summary of all adverse events
(Safety population)

| | | | 200 ug | | 400 ug | | 800 ug | |
|---|---|---|---|---|---|---|---|---|
| | Placebo | | VR004 | | VR004 | | VR004 | |
| | N | % | N | % | N | % | N | % |
| Subjects treated | 35 | | 35 | | 35 | | 35 | |
| With adverse events | 4 | 11% | 3 | 9% | 3 | 9% | 2 | 6% |
| With severe AEs | 0 | | 0 | | 0 | | 0 | |
| With serious AEs | 0 | | 0 | | 0 | | 0 | |
| Discontinued due to AE | 0 | | 0 | | 0 | | 0 | |

TABLE 7
Summary of treatment-related adverse events
(Safety population)

| | | | 200 ug | | 400 ug | | 800 ug | |
|---|---|---|---|---|---|---|---|---|
| | Placebo | | VR004 | | VR004 | | VR004 | |
| | N | % | N | % | N | % | N | % |
| Subjects treated | 35 | | 35 | | 35 | | 35 | |
| With adverse events | 2 | 6% | 2 | 6% | 3 | 9% | 2 | 6% |
| With severe AEs | 0 | | 0 | | 0 | | 0 | |
| With serious AEs | 0 | | 0 | | 0 | | 0 | |
| Discontinued due to AE | 0 | | 0 | | 0 | | 0 | |

TABLE 8
Treatment-related adverse events by body system
(Safety population)

| | | | 200 μg | | 400 μg | | 800 μg | |
|---|---|---|---|---|---|---|---|---|
| | Placebo | | Apo | | Apo | | Apo | |
| Body system/Preferred term | N | % | N | % | N | % | N | % |
| Subjects treated | 35 | | 35 | | 35 | | 35 | |
| Gastrointestinal disorders | 1 | 3% | 0 | | 0 | | 1 | 3% |
| Nausea | 0 | | 0 | | 0 | | 1 | 3% |
| Vomiting NOS | 1 | 3% | 0 | | 0 | | 0 | |
| Nervous system disorders | 1 | 3% | 1 | 3% | 0 | | 2 | 6% |
| Dizziness | 0 | | 1 | 3% | 0 | | 2 | 6% |
| Headache | 1 | 3% | 0 | | 0 | | 0 | |
| Respiratory, thoracic and | 2 | 6% | 1 | 3% | 3 | 9% | 0 | |
| mediastinal disorders | | | | | | | | |
| Cough | 1 | 3% | 1 | 3% | 0 | | 0 | |
| Dry throat | 1 | 3% | 0 | | 1 | 3% | 0 | |
| Nasal congestion | 0 | | 1 | 3% | 0 | | 0 | |
| Pharyngolaryngeal pain | 0 | | 0 | | 2 | 6% | 0 | |
| Sneezing | 0 | | 1 | 3% | 0 | | 0 | |

For each patient, blood samples were taken 70 minutes after inhalation. The blood samples were analyzed, and the blood levels for 400 and 800 microgram doses of apomorphine for each of the 34 patients that completed the test are set forth in table 9:

TABLE 9

| Patient ID | Apomorphine HCL 800 μg | Apomorphine HCL 400 μg |
|---|---|---|
| Sub 1 | 0.540 | 0.138 |
| Sub 2 | 0.829 | 0.293 |
| Sub 3 | 0.716 | 0.233 |
| Sub 4 | 0.456 | 0.256 |
| Sub 5 | 0.468 | 0.300 |
| Sub 6 | 0.656 | 0.274 |
| Sub 7 | 0.550 | 0.133 |
| Sub 8 | 0.740 | 0.424 |
| Sub 9 | 0.824 | 0.271 |
| Sub 10 | 0.415 | 0.153 |
| Sub 11 | 0.585 | 0.253 |
| Sub 12 | 0.570 | 0.240 |
| Sub 13 | 0.271 | 0.140 |
| Sub 14 | 0.563 | 0.398 |
| Sub 15 | 0.549 | 0.294 |
| Sub 16 | 0.367 | 0.171 |
| Sub 17 | 0.504 | 0.219 |
| Sub 19 | 0.756 | 0.000 |
| Sub 20 | 0.467 | 0.214 |
| Sub 21 | 0.646 | 0.207 |
| Sub 22 | 0.734 | 0.226 |
| Sub 23 | 0.648 | 0.263 |
| Sub 24 | 0.598 | 0.205 |
| Sub 25 | 0.384 | 0.188 |
| Sub 26 | 0.730 | 0.167 |
| Sub 27 | 0.437 | 0.174 |
| Sub 28 | 0.414 | 0.132 |
| Sub 29 | 1.040 | 0.109 |
| Sub 30 | 0.593 | 0.220 |
| Sub 31 | 1.471 | 0.126 |
| Sub 32 | 0.446 | 0.251 |
| Sub 33 | 0.501 | 0.244 |
| Sub 34 | 0.405 | 0.177 |
| Sub 35 | 0.808 | 0.213 |
| mean | 0.608 | 0.215 |
| median | 0.567 | 0.217 |

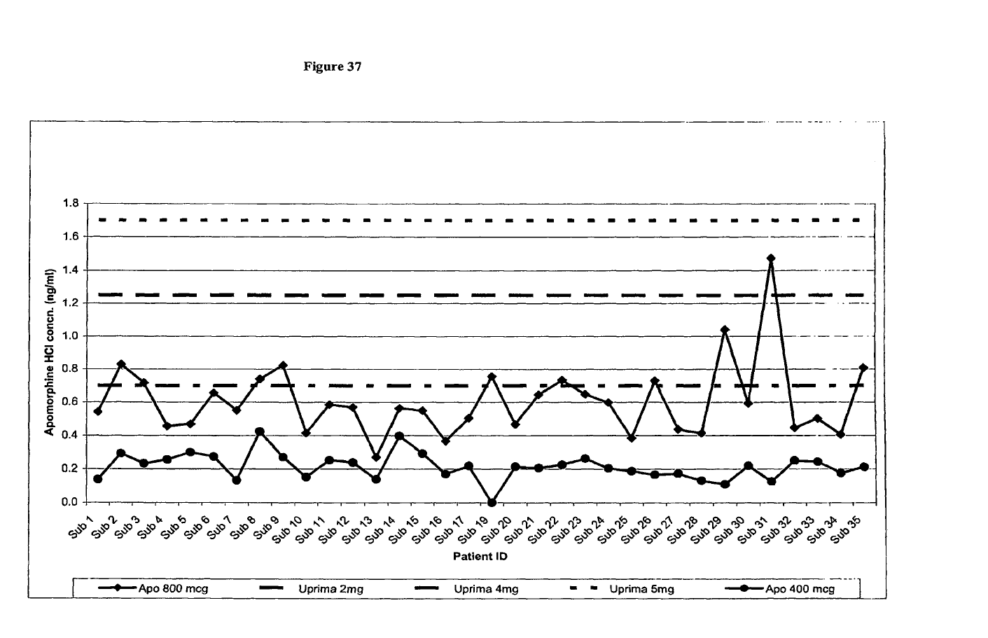

FIG. 37 shows a comparison of the blood levels at 70 minutes after dosing (T₇₀) for each patient for the 400 microgram dose and the 800 microgram dose. Also plotted is the known mean Cₘₐₓ of 2 mg (0.7 ng/ml), 4 mg (1.25 ng/ml), and 5 mg (1.7 ng/ml) of Uprima™ sublingual tablets. In this regard, 4 mg and 5 mg Uprima sublingual tablets are known to have unacceptable side effects. For example, the 4 mg Uprima sublingual tablets were found to have unacceptable clinical safety by the European Agency for the Evaluation of Medicinal Products (See EPAR (European Public Assessment Safety Report) 1945, Uprima, common name apomorphine hydrochloride, “Scientific Discussion”, pp. 25-27 (2002)).

The mean plasma levels at 70 minutes after dosing (T₇₀) at 400 μg and 800 μg were 0.22 and 0.61 ng/mL respectively. These T₇₀ levels are below those known to be efficacious (See EPAR 1945).

It should be noted that it was not feasible to take plasma samples at earlier time points because of the need to protect the privacy and dignity of the volunteers during the period that efficacy was evaluated. Moreover, it is believed that the process of drawing blood samples during the VSS period of the test would have affected to the ability of the patients to maintain an erection. It is therefore necessary to back-calculate the plasma concentration to the time when concentration was a maximum (Cₘₐₓ), as therapeutic (pharmacological) effects usually depend upon the value of Cₘₐₓ. Back-calculation procedures are well known in the art, and use a model based on the half-life of the drug in plasma. Inhalation absorption is known to be rapid and complete because of the large surface area and profuse blood supply of the lung. As this pattern of absorption is similar to that of intravenous dosing, it is reasonable to take the time immediately after dosing (T₀) as the approximate timepoint associated with Cₘₐₓ, and to use the half-life known for intravenous administration of apomorphine (41 minutes as cited by van der Geeste, R Clin. Neuropharmacol. 21 (3) (1998)).

Using this information, the correction factor based on the half-life of apomorphine is 3.26 (270/41). Applying this to the mean T₇₀ values of Table 9 yields estimated mean plasma levels at T₀ of 0.72 ng/ml for the 400 μs dose and 1.99 ng/mL for the 800 μg dose. These levels were expected to be efficacious based upon the above-referenced EPAR 1945, which is consistent with the clinical data of Table 4 above.

In addition to the clinical data described above in connection with Tables 6-8, the blood level data of Table 9 further supports the conclusion that the inhaled apomorphine in accordance with the embodiments of the present invention minimizes the risk of side effects.

First, therapeutic (pharmacological) effects are usually dependent upon the value of Cₘₐₓ. However, side-effects are often dependent upon the systemic exposure to the drug. Systemic exposure can be measured as the integral of the plasma level from time of administration until it returns to zero (i.e. the area under the curve AUC_{0 to ∝}). The measured values of Table 9 demonstrate that plasma levels fall rather rapidly to low values after dosing via inhalation in accordance with the invention. In contrast, absorption is much less rapid and complete by most other routes of administration. For example, EPAR 1945 reports that the elimination half-life for Uprima is 2.7 hours for a 2 mg sublingual dose, 4.2 hours for a 4 mg sublingual dose, 3.9 hours for a 5 mg sublingual dose, and 4.0 hours for a 6 mg sublingual dose. (EPAR 1945, “Scientific Discussion”, p. 12).

A second but equally important beneficial effect of the short half-life associated with the inhaled formulation is that the period in which therapeutic and any side-effects is short due to the short half-life of the formulation. Consequently, side-effects, if they occur, will be short lived, allowing the patient to resume normal activities such as driving.

The data of Table 9 and FIG. 37 also demonstrates that the intersubject variability of the formulation was low for each dose level for which plasma levels were measured. For example, the coefficient of variation was only 37%, demonstrating the consistency of the procedure and thereby minimizing the risk of under-dosing or over-dosing.

The data also indicates that doses can be readily matched to an individual subject. Referring to FIG. 37, it is apparent that the pair of values for an single subject tended to be associated (i.e. the intrasubject variability was lower than the intersubject variability). Consistency in plasma levels can therefore be expected on each occasion that a subject receives therapy. This should provide an opportunity for a subject to select a dose that is appropriate to him.

### Example 15

### Solution pMDI Formulations

A pMDI fomulation was prepared with the ingredients listed in the following table. The formulation can be placed in a 3M coated (Dupont 3200 200) canister with a Bespak BK630 series 0.22 mm actuator for subsequent delivery to the lungs of a patient as described above:

| | 200 ug Formulation | | |
|---|---|---|---|
| | Vol. | Amount | Percentage |
| Apomorphine | 0.0200 ml | 24 mg | 0.1931% w/w |
| HCl (Ex. 2) | | | |
| HFA134a | 6.45 ml | 7905 mg | 63.60% w/w |
| Water | 0.75 ml | 749 mg | 6.03% |
| Ethanol | 4.75 ml | 3751.50 mg | 30.18% |
| Total Formulation | | 12429.50 mg | |
| Weight | | | |
| Total Formulation | 11.97 ml | | |
| Volume | | | |
| Estimated dose of | | 200 ug/100 ul | |
| Apomorphine | | | |
| HCl | | | |

It is expected that this formulation can provide a Fine Particle Fraction of between 10% and 30%.

### Example 16

### Suspension pMDI

Suspension pMDIs were prepared with HFA227, HFA134a, and apomorphine hydrochloride in a 3M coated (Dupont 3200 200) canister with a Bespak BK630 series 0.22 mm actuator. Specifically, the following formulations were prepared:

| | Formulation A | | | Formulation B | | |
|---|---|---|---|---|---|---|
| | Amount | | Percentage | Amount | | Percentage |
| Apomorphine HCl | 26.7 | mg | 0.23% w/w | 104 | mg | 0.9% w/w |
| (Ex. 2) | | | | | | |
| HFA134a | 4229 | mg | 37.14% w/w | 4321.7 | mg | 37.4% w/w |
| HFA227 | 7129.7 | | 62.62% w/w | 7129.7 | mg | 61.7% w/w |
| Total Formulation | 11385.4 | mg | | 11555.4 | mg | |
| Weight | | | | | | |
| Total Formulation | 8.5 | ml | | 8.7 | ml | |
| Volume | | | | | | |
| (Estimated) | | | | | | |

| | | |
|---|---|---|
| Estimated dose of | 157 ug/50 μl | 600 ug/50 μl |
| Apomorphine HCl | | |

Formulation B was tested with an Anderson Cascade Impactor over 10 discharges. The results were as follows, each value being an average of the 10 discharges:

| Metered Dose: | 517.43 ug |
|---|---|
| Delivered Dose: | 470.96 ug |
| MMAD: | 3.47 um |
| Fine Particle Dose: | 314.140 ug |
| Fine Particle Fraction: | 66.7% |

wherein a fine particle is defined as a particle having a diameter of less than or equal to 5 microns.

### Example 17

### 400 μg Apomorphine Hydrochloride Capsule For Use in Cyclohaler

Five 400 μg apomorphine hydrochloride capsules were prepared and tested in a Cyclohaler inhaler (available from Miat) in an ACI (U.S.P. 26, Chapter 601, Apparatus 3) configured for operation at 100 l·min-1. Each capsule had a fill weight of 25 mg, and included the following components:

Weight

| Component | (g) | % (w/w) |
|---|---|---|
| Pharmatose 150M | 127.725 | 85.15 |
| (DMV Pharma) | | |
| Sorbolac 400 | 12.375 | 8.25 |
| (Meggle Pharma) | | |
| Micronised Leucine | 7.500 | 5.00 |
| (As described in Example 10) | | |
| Apomorphine Hydrochloride | 2.400 | 1.60 |
| (d₅₀ = 1.453 microns) | | |
| (As described in FIG. 23(b)) | | |

In this regard, Pharmatose 150M, available from DMV Pharma, comprises lactose with the following particle size distribution (according to DMV Pharma literature): 100% less than 315 microns, at least 85% less than 150 microns, at least 70% less than 100 microns, and at least 50% less than 45 microns. Sorbolac 400, available from Meggle Pharma comprises lactose with the following particle size distribution (according to Meggle Pharma literature): 100% less than 100 microns, at least 99% less than 63 microns, and at least 96% less than 32 microns.

### Preparation of Pre-Blend

The Pharmatose, Sorbolac and Leucine were layered in the mixing bowl so that the leucine was sandwiched between the Sorbolac, which in turn was sandwiched between the Pharmatose. The powders were blended for 60 seconds at 2000 rpm using the Retsch Grindomix High Shear Mixer described above. The pre-blend was rested for 1 hour before further use.

### Preparation of Final Blend

The apomorphine hydrochloride was sandwiched between the pre-blend in the mixing bowl. Blending was carried out for 10 minutes at 2000 rpm using the Grindomix mixer. The blend was then passed through a 212 μm sieve.

Thereafter, the final blend was placed in capsules, each capsule having a fill weight of 25 mg. The capsules were then placed in a cyclohaler and tested in an ACI (U.S.P. 26, Chapter 601, Apparatus 3), with the data analyzed via the CITDAS described above, providing the following results:

| | |
|---|---|
| Delivered Dose (%) | 81% |
| (100 * Delivered Dose/Total Dose) | |
| Fine Particle Fraction | 67% |
| (percent of the delivered dose ≦5 microns) | |
| % Fine Particle Dose | 55% |
| (percent of the total dose ≦5 microns) | |
| MMAD | 2.3 microns |
| Fine Particle Dose | 220 μg |
| % Ultrafine Particle Dose | 44% |
| (percent of the total dose ≦3 microns) | |
| Ultrafine Particle Dose | 175 μg |
| Ultrafine Particle Fraction | 53% |

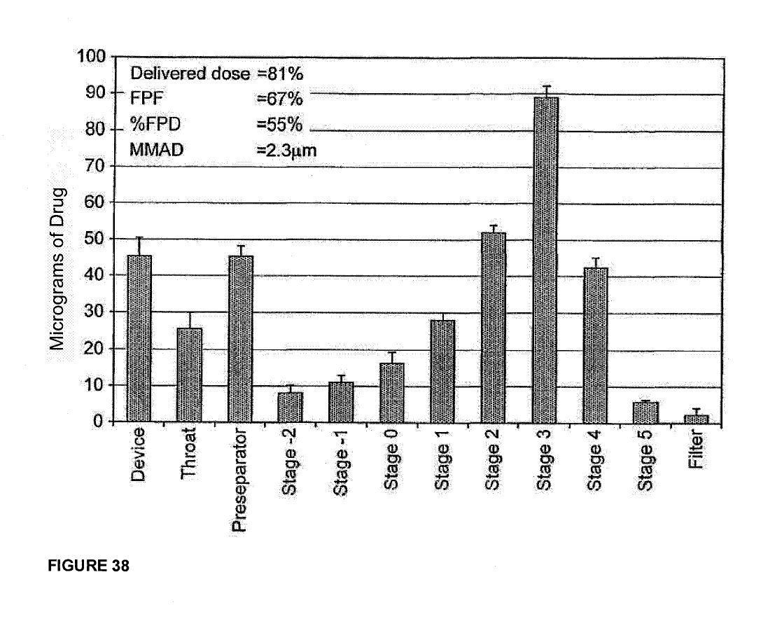

FIG. 38 illustrates the average amount (in micrograms) of drug that was delivered to each of the components of the ACI, and retained in the device. Thus, for example, the Ultrafine Particle Dose can be produced from this data by the CITDAS package.

### Example 18

### 400 μg Apomorphine Hydrochloride 2 Mg Blister

Five 400 μg apomorphine hydrochloride blisters were prepared and tested in the inhaler of example 5 in an ACI (USP 26, Chapter 601, Apparatus 3) configured for operation at 60 l·min⁻¹. Each blister had a fill weight of 2 mg, and included the following components:

Weight

| Component | (g) | % (w/w) |
|---|---|---|
| Respitose 45-63 μm sieve | 120 | 80 |
| (As described in Example 1) | | |
| Apomorphine Hydrochloride | 30 | 20 |
| (d₅₀ = 1.453 micrograms) | | |
| (As described in FIG. 23(b)) | | |

The Apomorphine hydrochloride was sandwiched between the Respitose in the mixing bowl as generally described in Examples 2(a) and 2(b). The powders were blended for 5 mins at 2000 rpm using the Grindomix mixer. The blend was then passed through a 212 μm sieve. Thereafter, the blend was placed in blister, each blister having a fill weight of 2 mg. The blisters were then placed in the inhaler of Example 5 and tested in an ACI (U.S.P. 26, Chapter 601, Apparatus 3), with the data analyzed via the CITDAS described above, providing the following results:

| | |
|---|---|
| Delivered Dose (%) | 89% |
| (100 * Delivered Dose/Total Dose) | |
| Fine Particle Fraction | 81% |
| (percent of the delivered dose ≦5 microns) | |
| % Fine Particle Dose | 72% |
| (percent of the total dose ≦5 microns) | |
| MMAD | 1.70 microns |
| Fine Particle Dose | 288 μg |
| % Ultrafine Particle Dose | 67% |
| (percent of the total dose ≦3 microns) | |
| Ultrafine Particle Dose | 266 μg |
| Ultrafine Particle Fraction | 75% |
| (percent of the delivered dose ≦3 microns) | |

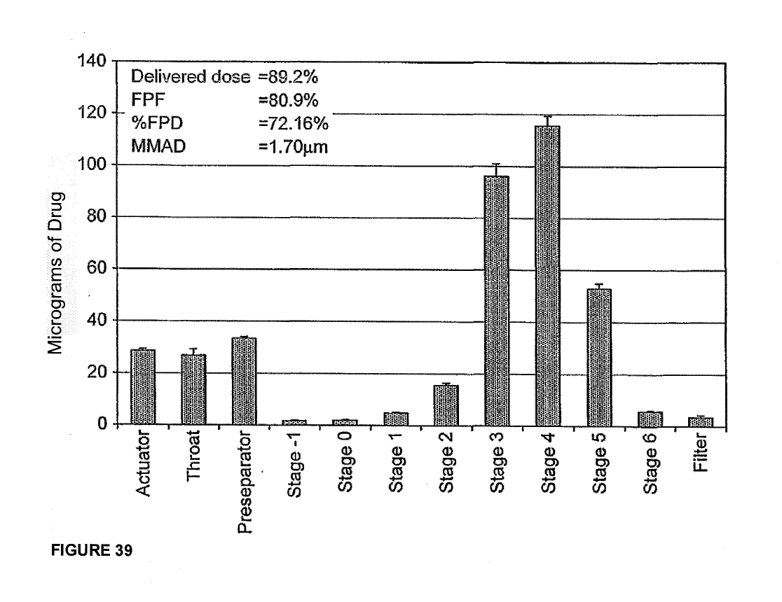

FIG. 39 illustrates the average amount (in micrograms) of drug that was delivered to the components of the ACI, and left in the device. Thus, for example, the ultrafine particle dose can be produced from this data using the CITDAS package.

It should be noted that the MMAD of 1.70 microns generated from the ACI data is remarkably fine, and very close to the median diameter determined by laser light diffraction, for this batch of apomorphine hydrochloride (1.453 microns as reported FIG. 23(b)). This indicates that the inhaler is efficiently reducing the drug to, or close to, its primary particles, rather than agglomerate. This is highly unusual for an inhaler. For example, when the same batch of apomorphine hydrochloride (i.e. in particle size) was delivered with the Cyclohaler of Example 17, a larger MMAD of 2.3 microns was measured, indicating that this formulation and device was not as efficient at eliminating agglomerates.

When compared with the formulation and inhaler of Example 17, the formulation and inhaler of Example 18 also provided a superior delivered dose (89.2% vs 81%), fine particle fraction (81% vs 67%), % fine particle dose (72% vs 55%) and % ultrafine particle dose (67% vs 44%).

It is also apparent from the above data that the formulation and inhaler of Example 18 produces an ultra-fine particle fraction (≦3 μm) of more than 70%. While a fine particle fraction (≦5 microns) can be considered acceptable for local delivery, it is believed that for systemic delivery, even finer particles are needed, because the drug must reach the alveoli to be absorbed into the bloodstream. As such an ultrafine particle fraction in excess of 70% is particularly advantageous.

The above referenced data indicates that the preferred inhaler in accordance with the present invention, is particularly efficient when combined with the preferred formulation in accordance with the present invention.

It should also be noted that both the formulation of Example 17 (with the cyclohaler) and the formulation of Example 18 (with the preferred inhaler), provide significantly better performance than the suspension pMDI of Example 15, which had an MMAD of 3.47, an FPF of 66.7, and a % Fine Particle Dose of 52.4%.

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

1. A method for treating sexual dysfunction in a human via inhalation, comprising:
inhaling a dose of a powder composition, the dose of the powder comprising from about 100 to about 1600 micrograms of apomorphine or a pharmaceutically acceptable salt or ester thereof (based on the weight of the hydrochloride salt), wherein the inhalation of the dose of apomorphine provides a therapeutic effect in less than about nine minutes, but is not associated with the adverse side effects normally associated with the administration of apomorphine.

2. The method of claim 1, wherein the sexual dysfunction is erectile dysfunction.

3. The method of claim 1, wherein the sexual dysfunction is female sexual dysfunction.

4. The method of claim 1, wherein the erectile dysfunction is psychogenic.

5. The method of claim 1, wherein the erectile dysfunction is organic.

6. The method of claim 1, wherein the dose comprises from about 200 micrograms to about 1600 of apomorphine or a pharmaceutically acceptable salt or ester thereof (based on the weight of the hydrochloride salt).

7. The method of claim 1, wherein the dose comprises from about 300 micrograms to about 1200 of apomorphine or a pharmaceutically acceptable salt or ester thereof (based on the weight of the hydrochloride salt).

8. The method of claim 1, wherein the dose comprises from about 400 micrograms to about 800 of apomorphine or a pharmaceutically acceptable salt or ester thereof (based on the weight of the hydrochloride salt).

9. The method of claim 8, wherein the sexual dysfunction is erectile dysfunction.

10. The method of claim 1, wherein the dose comprises from about 400 micrograms to about 1200 micrograms of apomorphine or a pharmaceutically acceptable salt or ester thereof (based on the weight of the hydrochloride salt).

11. The method of claim 10, wherein the sexual dysfunction is erectile dysfunction.

12. The method of claim 1, wherein the powder composition further includes a carrier material.

13. The method of claim 12, wherein the dose includes from about 400 to about 800 micrograms of apomorphine hydrochloride.

14. The method of claim 13, wherein the dose provides, in vivo, a mean Cₘₐₓ of from about 0.7 ng/ml to about 2 ng/ml.

15. The method of claim 14, wherein the dose provides, in vivo, a mean plasma level of said apomorphine at seventy minutes after administration of from about 0.2 ng/ml to about 0.6 ng/ml.

16. The method of claim 13, wherein the apomorphine is apomorphine hydrochloride and at least 99% of said apomorphine hydrochloride has a particle size of 5 microns or less.

17. The method of claim 1, wherein the dose comprises a powder composition which further includes apomorphine or a pharmaceutically acceptable salt or ester thereof and an anti-adherent material.

18.-23. (canceled)

24. The method of claim 12, wherein the carrier material is lactose and the apomorphine is apomorphine hydrochloride.

25.-29. (canceled)

30. The method of claim 12 wherein the powder composition further includes a force control additive.

31. The method of claim 30, wherein the force control additive is provided in an amount from about 0.15% to about 5% of the composition, by weight.

32. The method of claim 30, wherein the force control additive is selected from the group consisting of leucine, magnesium stearate, lecithin, and sodium stearyl fumarate.

33. The method of claim 30, wherein the force control additive includes leucine.

34. A method for treating sexual dysfunction in a human via inhalation, comprising inhaling a dose of a powder composition into the lungs of a patient, the dose of the powder composition delivering, in vitro, a fine particle dose of from about 100 micrograms to about 1600 micrograms of apomorphine or a pharmaceutically acceptable salt or ester thereof (based on the weight of the hydrochloride salt), when measured by a Multistage Liquid Impinger, United States Pharmacopeia 26, Chapter 601 Apparatus 4 (2003), wherein the inhalation of the dose of apomorphine provides a therapeutic effect in less than about nine minutes, but is not associated with the adverse side effects normally associated with the administration of apomorphine.

35. The method of claim 34, wherein the dose delivers, in vitro, a fine particle dose of from about 200 micrograms to about 1000 micrograms of said apomorphine when measured by a Multistage Liquid Impinger, United States Pharmacopeia 26, Chapter 601 Apparatus 4 (2003).

36. The method of claim 34, wherein the dose delivers, in vitro, a fine particle dose of from about 200 micrograms to about 800 micrograms of said apomorphine when measured by a Multistage Liquid Impinger, United States Pharmacopeia 26, Chapter 601 Apparatus 4 (2003).

37. The method of claim 34, wherein the dose delivers, in vitro, a fine particle dose of from about 200 micrograms to about 600 micrograms of said apomorphine when measured by a Multistage Liquid Impinger, United States Pharmacopeia 26, Chapter 601 Apparatus 4 (2003).

38. The method of claim 34, wherein the dose delivers, in vitro, a fine particle dose of from about 200 to about 400 micrograms of said apomorphine when measured by a Multistage Liquid Impinger, United States Pharmacopeia 26, Chapter 601 Apparatus 4 (2003).

39.-46. (canceled)

47. The method of claim 1, wherein the step of inhaling comprises:
entraining the powder composition in a gas flow upstream from an inlet port of a vortex chamber having a substantially circular cross-section,
directing the gas flow through the inlet port into the vortex chamber in a tangential direction;
directing the gas flow through the vortex chamber so as to aerosolise the powder composition; and
directing the gas flow with the powder composition out of the vortex chamber in an axial direction through an exit port, wherein a velocity of the gas flow at a distance of 300 mm outside of the exit port is less than a velocity of the gas flow at the inlet port.

48. The method of claim 1, wherein the powder composition comprises agglomerated particles, and the step of inhaling comprises:
entraining the agglomerated particles in a gas flow upstream from an inlet port of a vortex chamber,
directing the gas flow through the inlet port into the vortex chamber;
depositing the agglomerated particles onto one or more walls of the vortex chamber;
applying, via the gas flow through the vortex chamber, a shear to the deposited agglomerated particles to deagglomerate said particles,
directing the gas flow, including the deagglomerated particles, out of the vortex chamber, wherein a velocity of the gas flow at a distance of 300 mm outside of the exit port is less than a velocity of the gas flow at the inlet port.

49. The method of claim 12, wherein the carrier material has an average particle size of from about 40 microns to about 70 microns, and at least 90% of said apomorphine having a particle size of 5 microns or less.

50. The method of claim 49, wherein the powder composition comprises agglomerated particles, and the step of inhaling comprises:
entraining the agglomerated particles in a gas flow,
depositing the agglomerated particles onto one or more surfaces;
applying, via the gas flow, a shear to the deposited agglomerated particles to deagglomerate said particles.

51. The method of claim 1, wherein the step of inhaling comprises:
generating an air flow through an inlet port of a chamber, the air flow having entrained therein the powder composition;
directing the air flow through the chamber, the chamber having an axis and a wall curved about the axis, the air flow rotating about the axis; and
directing the air flow through an exit port of the chamber,
wherein a direction of the air flow through the inlet port is tangential to the wall, and a direction of the air flow through the exit port is parallel to the axis,
and wherein a cross-sectional area of the air flow through the chamber is in a plane normal to the air flow and decreases with increasing distance from the inlet port.

52-68. (canceled)

69. The method of claim 1, wherein the powder composition comprises agglomerated particles, and the step of inhaling comprises:
entraining the agglomerated particles in a gas flow upstream from an inlet port of a vortex chamber,
directing the gas flow through the inlet port into the vortex chamber;
depositing the agglomerated particles onto one or more walls of the vortex chamber;
applying, via the gas flow through the vortex chamber, a shear to the deposited agglomerated particles to deagglomerate said particles,
directing the gas flow, including the deagglomerated particles, out of the vortex chamber to provide an ultrafine particle fraction, when measured by an Andersen Cascade Impactor, United States Pharmacopeia 26, Chapter 601 Apparatus 3 (2003), of at least about 70%.

70. The method of claim 1, wherein the step of inhaling comprises inhaling a dose having an ultrafine particle fraction, when measured by an Andersen Cascade Impactor, United States Pharmacopeia 26, Chapter 601 Apparatus 3 (2003), of at least about 70%.

71. (canceled)

72. The method of claim 1, wherein the step of inhaling comprises inhaling a dose having a fine particle fraction, when measured by an Andersen Cascade Impactor, United States Pharmacopeia 26, Chapter 601 Apparatus 3 (2003), of at least about 80%.

73. (canceled)
